(19) United States
(12) Patent Application Publication
(10) Pub. No.: US 2024/0423443 A1
(43) Pub. Date: Dec. 26, 2024

(54) ENDOSCOPE CONTROL SYSTEM AND ENDOSCOPE CONTROL METHOD

(71) Applicant: OLYMPUS MEDICAL SYSTEMS CORP., Tokyo (JP)

(72) Inventors: Fuyuki ONEYAMA, Tokyo (JP); Hirokazu NISHIMURA, Tokyo (JP)

(73) Assignee: OLYMPUS MEDICAL SYSTEMS CORP., Tokyo (JP)

(21) Appl. No.: 18/828,572

(22) Filed: Sep. 9, 2024

Related U.S. Application Data
(63) Continuation of application No. PCT/JP2022/012386, filed on Mar. 17, 2022.

(51) Int. Cl. A61B 1/00; A61B 1/005; G06V 10/764

(52) U.S. Cl. CPC A61B 1/00006; A61B 1/00045; A61B 1/0051; G06V 10/764; G06V 2201/031

(57) ABSTRACT

An image acquisitor acquires an image of a lumen imaged by an endoscope. An image classifier classifies the acquired lumen image as one of a plurality of types. When the lumen image is classified as a first type, an operation detail determinator determines an insertion operation detail of the endoscope using an insertion operation selection model generated by machine learning. When the lumen image is classified as a second type, the operation detail determinator determines the insertion operation detail of the endoscope using an algorithm for determining the insertion operation detail.

### CROSS-REFERENCE TO RELATED APPLICATIONS

This application is based upon and claims the benefit of priority from the International Application No. PCT/JP2022/012386, filed on Mar. 17, 2022, the entire contents of which are incorporated.

### BACKGROUND

### 1. Technical Field

The present disclosure relates to a technique for determining an operation detail of an endoscope from an image imaged by the endoscope.

### 2. Description of the Related Art

In endoscopic observation, a flexible elongated insertion portion is inserted deep inside a subject, and the inside of the subject is imaged. In recent years, studies have been conducted on techniques to automate operations on an insertion portion. For example, in an electronic endoscope apparatus in which an insertion portion is provided with a bending portion that can be bent upward, downward, leftward, and rightward, JP 3645223 B2 discloses a technique for controlling the bending angle of the bending portion such that the distal end of the insertion portion faces the center of the lumen being imaged.

WO 2021/49475 A discloses an endoscope system that determines one or more operation details from among a plurality of operation details using an operation selection model generated by machine learning, and controls a movement of an endoscope based on the determined one or more operation details. The operation selection model is generated by machine learning using, as training data, an image for learning, which is an endoscopic image imaged in the past, and a label that is assigned to the image for learning and that indicates an operation detail of an endoscope that has imaged the image for learning. The operation detail is determined by inputting input data acquired from the imaged endoscopic image into the operation selection model.

In recent years, as a technique related to deep learning, a method of estimating information in a depth direction from an image has been proposed (Lei He, Guanghui Wang and Zhanyi Hu, “Learning Depth from Single Images with Deep Neural Network Embedding Focal Length”, 27 Mar. 2018 <URL: https://arxiv.org/pdf/1803.10039.pdf>), and research to generate information in the depth direction from the endoscopic image has also been conducted (Faisal Mahmood, Richard Chen, Nicholas J. Durr, “Unsupervised Reverse Domain Adaptation for Synthetic Medical Images via Adversarial Training”, 29 Nov. 2017 <URL: https://arxiv.org/pdf/1711.06606.pdf>).

FIG. 1 is a schematic diagram of the large intestine. The site such as the sigmoid colon, the splenic flexure, and the hepatic flexure has a greatly bent shape. Particularly, the sigmoid colon is not fixed and moves significantly. Although the splenic flexure and the hepatic flexure are fixed, the flexures move due to insufficient fixation. Accordingly, the difficulty of inserting an endoscope into these sites is considered to be high. Even in a situation where the intestinal tract has a complicated shape or the lumen is narrowed due to stenosis or the like, the difficulty of inserting the endoscope increases. Therefore, in the automatic insertion control of the endoscope, it is desired to develop a technique for safely passing the distal end of the endoscope through a site having a high difficulty of insertion. In addition, in an endoscopic examination in which a physician manually inserts an endoscope, presentation of information for safely passing the distal end of the endoscope through a site having a high difficulty of insertion is considered to be useful for improving the skill of the physician.

### SUMMARY

The present disclosure has been made in view of the above circumstances, and an object of the present disclosure is to provide a technique for generating appropriate operation detail of an endoscope according to a situation.

An endoscope control system according to one aspect of the present disclosure is an endoscope control system that determines an operation detail of an endoscope, the endoscope control system including one or more processors having hardware. The one or more processors acquire an endoscopic image imaged by the endoscope, classify the acquired endoscopic image as any one of a plurality of types, determine the operation detail of the endoscope using an operation selection model generated by machine learning when the endoscopic image is classified as a first type, and determine the operation detail of the endoscope using an algorithm for determining the operation detail when the endoscopic image is classified as a second type.

An endoscope control method according to another aspect of the present disclosure acquires an endoscopic image imaged by the endoscope, classifies the acquired endoscopic image as any one of a plurality of types, determines the operation detail of the endoscope using an operation selection model generated by machine learning when the endoscopic image is classified as a first type, and determines the operation detail of the endoscope using an algorithm for determining the operation detail when the endoscopic image is classified as a second type.

Note that arbitrary combinations of the above components and modifications of the expressions of the present disclosure among methods, apparatuses, systems, storage media, computer programs, and the like are also effective as aspects of the present disclosure.

### BRIEF DESCRIPTION OF THE DRAWINGS

Embodiments will now be described, by way of example only, with reference to the accompanying drawings which are meant to be exemplary, not limiting, and wherein like elements are numbered alike in several Figures, in which:

FIG. 1 is a schematic diagram of the large intestine;

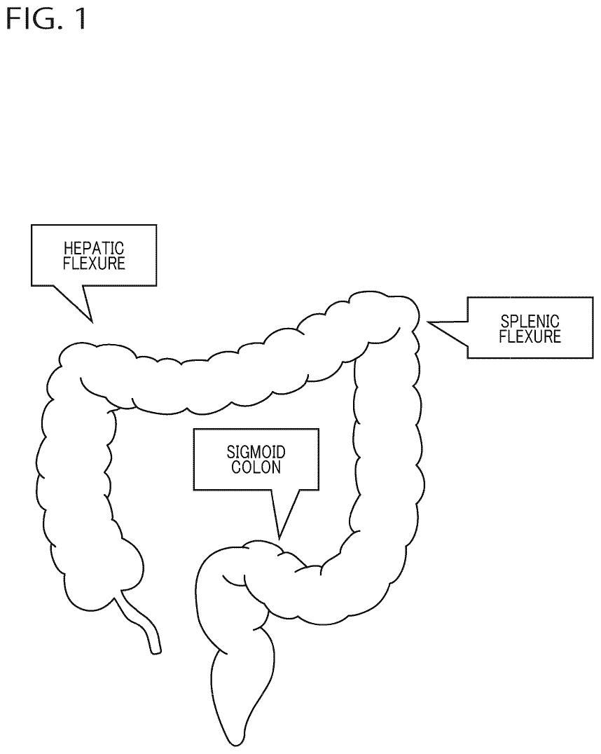

FIG. 2 is a diagram illustrating a configuration of an endoscope control system;

FIG. 3 is a diagram illustrating functional blocks of the endoscope control system;

FIGS. 4A and 4B are diagrams showing examples of endoscopic images;

FIG. 5 is a schematic diagram of the large intestine;

FIGS. 6A and 6B are diagrams showing examples of endoscopic images;

FIGS. 7A and 7B are diagrams showing examples of endoscopic images;

FIG. 8 is a diagram illustrating functional blocks of a controller;

FIG. 9 is a diagram showing an example of training data;

FIG. 10 is a diagram showing another example of training data;

FIG. 11 is a diagram showing another example of training data;

FIG. 12 is a diagram showing another example of training data;

FIG. 13 is a diagram showing another example of training data;

FIG. 14 is a diagram showing another example of training data;

FIG. 15 is a diagram showing another example of training data;

FIG. 16 is a diagram showing another example of training data;

FIG. 17 is a diagram showing another example of training data;

FIG. 18 is a diagram showing another example of training data;

FIG. 19 is a diagram showing another example of training data;

FIG. 20 is a flowchart for determining operation details of the endoscope;

FIGS. 21A and 21B are diagrams showing examples of images for learning to which a third type label is assigned;

FIGS. 22A and 22B are diagrams showing examples of images for learning to which a fourth type label is assigned;

FIG. 23A is a diagram showing an endoscopic image, and FIG. 23B is a diagram showing a segmentation result image;

FIG. 24A is a diagram showing an endoscopic image, and FIG. 24B is a diagram showing a segmentation result image;

FIG. 25A is a diagram showing an endoscopic image, and FIG. 25B is a diagram showing a depth estimation result image;

FIG. 26 is a flowchart illustrating a procedure in which the distal end of the endoscope passes through a bending portion;

FIG. 27A is a diagram showing an endoscopic image, FIG. 27B is a diagram showing a segmentation result image, and FIG. 27C is a diagram showing a depth estimation result image;

FIG. 28 is a diagram for explaining a method of obtaining the center of gravity of a group of pixels;

FIG. 29A is a diagram showing an endoscopic image, FIG. 29B is a diagram showing a segmentation result image, and FIG. 29C is a diagram showing a depth estimation result image;

FIG. 30A is a diagram showing an endoscopic image, FIG. 30B is a diagram showing a segmentation result image, and FIG. 30C is a diagram showing a depth estimation result image;

FIG. 31A is a diagram showing an endoscopic image, FIG. 31B is a diagram showing a segmentation result image, and FIG. 31C is a diagram showing a depth estimation result image;

FIG. 32 is a flowchart illustrating a procedure in which the distal end of the endoscope passes through the stenosis part;

FIG. 33 is a flowchart illustrating a procedure in which the distal end of the endoscope passes through the multiple diverticulum;

FIG. 34 is a diagram showing a fourth type of endoscopic image that includes one or more diverticula;

FIGS. 35A and 35B are diagrams showing images in which a lumen region and a fold edge region are superimposed on an endoscopic image; and

FIG. 36 is a diagram showing an example of an endoscopic image.

### DETAILED DESCRIPTION

The disclosure will now be described by reference to the preferred embodiments. This does not intend to limit the scope of the present disclosure, but to exemplify the disclosure.

Hereinafter, embodiments of the present disclosure will be explained with reference to the drawings.

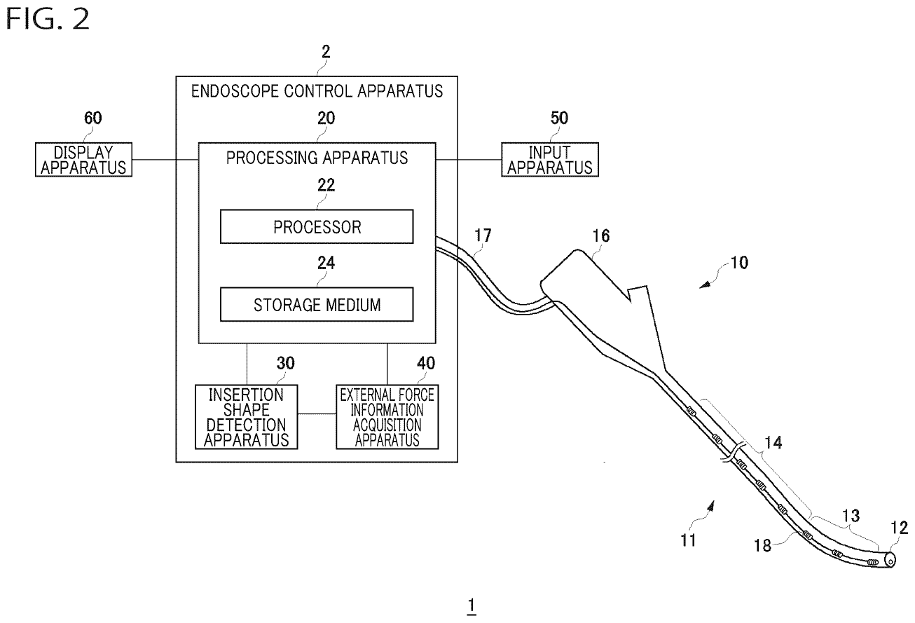

FIG. 2 shows the configuration of an endoscope control system 1 according to an embodiment. The endoscope control system 1 is provided in an endoscopic examination room and includes an endoscope control apparatus 2, an endoscope 10, an input apparatus 50, and a display apparatus 60. The endoscope control apparatus 2 includes a processing apparatus 20, an insertion shape detection apparatus 30, and an external force information acquisition apparatus 40, and has a function of automatically operating the movement of the endoscope 10 inserted into the body of the subject. The automatic operation of the endoscope 10 is performed by the processing apparatus 20 including one or more processors 22 and a storage medium 24.

The input apparatus 50 is an input interface operated by a user, and is formed to output an instruction according to the user's operation to the processing apparatus 20. The input apparatus 50 may include an operation apparatus such as a mouse, a keyboard, or a touch panel. The display apparatus 60 is a device that displays, on a screen, an endoscopic image or the like output from the processing apparatus 20, and may be a liquid crystal display or an organic EL display.

The endoscope 10 includes an imager including a solid-state imaging device (e.g. a CCD image sensor or a CMOS image sensor). The solid-state imaging device converts incident light into an electrical signal and outputs the electrical signal to the processing apparatus 20. The processing apparatus 20 has a signal processer that performs signal processing such as A/D conversion and noise removal on an imaging signal photoelectrically converted by the solid-state imaging device, and generates an endoscopic image. The signal processer may be installed on the endoscope 10 side, and the endoscope 10 may generate the endoscopic image. The processing apparatus 20 displays video imaged by the endoscope 10 on the display apparatus 60 in real time.

The endoscope 10 includes an insertion portion 11 to be inserted into the subject, an operation unit 16 provided on the base end side of the insertion portion 11, and a universal cord 17 extending from the operation unit 16. The endoscope 10 is detachably connected to the processing apparatus 20 by a scope connector (not illustrated) provided at an end of the universal cord 17.

The insertion portion 11 having an elongated shape has a rigid distal end 12, a bending portion 13 formed to be freely bendable, and a long flexible tube 14 having flexibility, in order from the distal end side to the base end side. Inside the distal end 12, the bending portion 13, and the flexible tube 14, a plurality of source coils 18 is arranged at predetermined intervals along the longitudinal direction of the insertion portion 11. The source coils 18 generate a magnetic field according to a coil drive signal supplied from the processing apparatus 20.

When a user such as a physician operates a release switch of the operation unit 16 while the endoscope 10 is being inserted into the subject, the processing apparatus 20 captures an endoscopic image and transmits the endoscopic image to an image server (not illustrated) for recording. The release switch may be provided on the input apparatus 50. A light guide (not illustrated) for transmitting illumination light supplied from the processing apparatus 20 so as to illuminate the inside of the subject is provided inside the endoscope 10. The distal end 12 is provided with an illumination window for emitting the illumination light transmitted by the light guide to the subject and an imager for imaging the subject at a predetermined cycle and outputting an imaging signal to the processing apparatus 20.

In the endoscope control system 1 according to the embodiment, the processing apparatus 20 automatically operates the endoscope 10 so as to automatically control the movement of the endoscope 10 inside the subject. Alternatively, it is also possible for the user to hold the operation unit 16 and manually operate the endoscope 10.

The operation unit 16 may include an operation member for the user to operate the endoscope 10. The operation unit 16 includes at least an angle knob for bending the bending portion 13 in eight directions that intersect with the longitudinal axis of the insertion portion 11.

The basic operations on the endoscope 10 are shown as follows:
“Advancement operation (push operation)” for moving the insertion portion 11 forward
“Retraction operation (pull operation)” for moving the insertion portion 11 backward
“Angle operation” for bending the bending portion 13
“Twist operation” for rotating the insertion portion 11 around the insertion axis
“Air feeding operation” for insufflating air: gas in a forward direction of the distal end 12
“Water feeding operation” for infusing water: liquid in a forward direction of the distal end 12
“Suction operation” for suctioning objects such as tissue fragments that are present near the distal end 12
“Search operation” for searching the center of the lumen by bending the bending portion 13 in multiple directions so as to point the distal end 12 in multiple directions

In the embodiment, the up-and-down direction of the distal end 12 is set as a direction orthogonal to the insertion axis of the insertion portion 11, and is also set as a direction corresponding to the vertical direction of the solid-state imaging device provided in the imager. Furthermore, the crosswise direction of the distal end 12 is set as a direction orthogonal to the insertion axis of the insertion portion 11 and is also set as a direction corresponding to the horizontal direction of the solid-state imaging device provided in the imager. Therefore, in the embodiment, the up-and-down direction of the distal end 12 matches the up-and-down direction of an endoscopic image output from a signal processer 220, and the crosswise direction of the distal end 12 matches the crosswise direction of the endoscopic image.

The processing apparatus 20 is detachably connected to each structure of the insertion shape detection apparatus 30, the external force information acquisition apparatus 40, the input apparatus 50, and the display apparatus 60. The processing apparatus 20 receives an instruction input by the user through the input apparatus 50 and performs processing that corresponds to the instruction. Further, the processing apparatus 20 acquires an imaging signal periodically output from the endoscope 10 and displays an endoscopic image on the display apparatus 60.

The insertion shape detection apparatus 30 has a function of detecting a magnetic field generated by each of the plurality of source coils 18 provided in the insertion portion 11 and acquiring the position of each of the plurality of source coils 18 based on the intensity of the detected magnetic field. The insertion shape detection apparatus 30 generates insertion shape information indicating the acquired positions of the plurality of source coils 18, and outputs the insertion shape information to the processing apparatus 20 and the external force information acquisition apparatus 40.

The external force information acquisition apparatus 40 stores data on the curvature (or radius of curvature) and bending angle of a predetermined plurality of positions of the insertion portion 11 in a state where no external force is applied and data on the curvature (or radius of curvature) and bending angle of the predetermined plurality of positions of the insertion portion 11 obtained in a state where a predetermined external force is applied to an arbitrary position of the insertion portion 11 from any conceivable direction. The external force information acquisition apparatus 40 identifies the positions of the plurality of source coils 18 provided in the insertion portion 11 based on the insertion shape information output from the insertion shape detection apparatus 30 and acquires the curvature (or radius of curvature) and the bending angle at each position of the plurality of source coils 18. The external force information acquisition apparatus 40 may acquire external force information indicating the magnitude and direction of the external force at each position of the plurality of source coils 18 from the acquired curvature (or radius of curvature) and bending angle and from various data stored in advance. The external force information acquisition apparatus 40 outputs the acquired external force information to the processing apparatus 20.

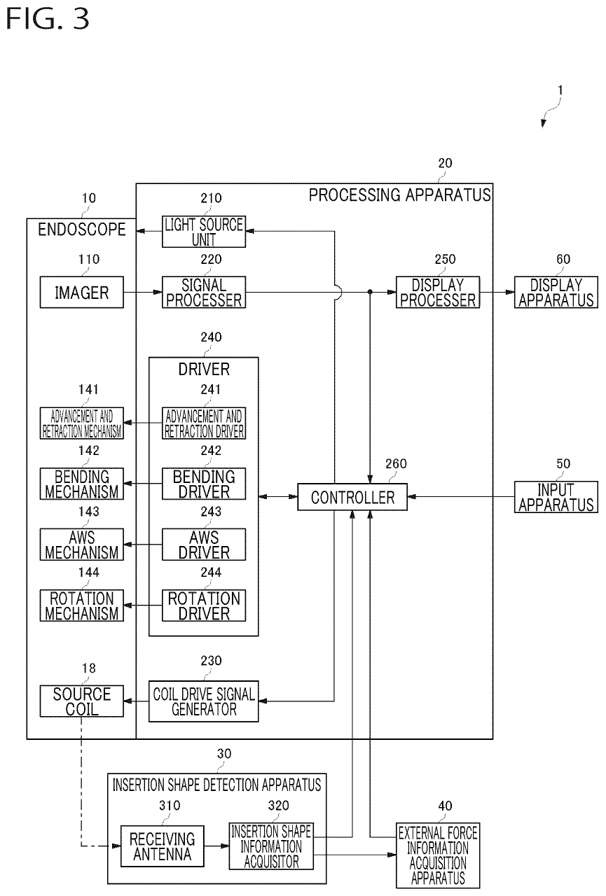

FIG. 3 illustrates functional blocks of the endoscope control system 1 according to the embodiment. The endoscope control system 1 includes an endoscope 10, a processing apparatus 20, an insertion shape detection apparatus 30, an external force information acquisition apparatus 40, an input apparatus 50, and a display apparatus 60.

The endoscope 10 includes a source coil 18, an imager 110, an advancement and retraction mechanism 141, a bending mechanism 142, an AWS mechanism 143, and a rotation mechanism 144. The advancement and retraction mechanism 141, the bending mechanism 142, the AWS mechanism 143, and the rotation mechanism 144 forms a movement mechanism in the endoscope 10.

The imager 110 has an observation window through which return light from the subject illuminated by illumination light enters, and a solid-state imaging device (e.g. a CCD image sensor or a CMOS image sensor) that images the return light and outputs an imaging signal.

The advancement and retraction mechanism 141 has a mechanism for realizing the movement of advancing and retracting the insertion portion 11. For example, the advancement and retraction mechanism 141 may be formed having a pair of rollers respectively arranged at opposite positions across the insertion portion 11 and a motor for rotating the pair of rollers. The advancement and retraction mechanism 141 drives the motor in response to an advancement and retraction control signal output from the processing apparatus 20 so as to rotate the pair of rollers, thereby executing either one of the movement of advancing the insertion portion 11 and the movement of retracting the insertion portion 11.

The bending mechanism 142 has a mechanism for realizing the movement of bending the bending portion 13. For example, the bending mechanism 142 may be formed having a plurality of bending pieces provided in the bending portion 13, a plurality of wires connected to the plurality of bending pieces, and a motor for pulling the plurality of wires. The bending mechanism 142 drives the motor in response to a bending control signal output from the processing apparatus 20 so as to change the amount of pulling of the plurality of wires, thereby allowing for the bending of the bending portion 13 in any of eight directions that intersect with the longitudinal axis of the insertion portion 11.

An air feeding, water feeding, and suction (AWS) mechanism 143 has a mechanism for realizing air feeding, water feeding, and suction movements. For example, the AWS mechanism 143 may be formed having two pipelines: an air and water feeding pipeline and a suction pipeline provided inside the insertion portion 11, the operation unit 16, and the universal cord 17, and a solenoid valve that performs a movement of opening one of the two pipelines while closing the other.

When activating the solenoid valve to open the air and water feeding pipeline in response to an AWS control signal output from the processing apparatus 20, the AWS mechanism 143 causes fluid including at least one of water and air supplied from the processing apparatus 20 to circulate into the air and water feeding pipeline and then discharge the fluid from an outlet port formed in the distal end 12. Further, when activating the solenoid valve to open the suction pipeline in response to the AWS control signal output from the processing apparatus 20, the AWS mechanism 143 applies a suction force generated by the processing apparatus 20 to the suction pipeline and suctions an object that exists near a suction port formed at the distal end 12 by the suction force.

The rotation mechanism 144 has a mechanism for realizing a movement of rotating the insertion portion 11 using the insertion axis of the insertion portion 11 as the rotation axis. For example, the rotation mechanism 144 may be formed having a support member that rotatably supports the insertion portion 11 on the base end side of the flexible tube 14, and a motor for rotating the support member. The rotation mechanism 144 rotates the insertion portion 11 around the insertion axis by driving the motor in response to a rotation control signal output from the processing apparatus 20 so as to rotate the support member.

The insertion shape detection apparatus 30 includes a receiving antenna 310 and an insertion shape information acquisitor 320. The receiving antenna 310 is formed having a plurality of coils that detect a magnetic field generated by each of the plurality of source coils 18 in a three-dimensional manner. Upon detecting the magnetic field generated by each of the plurality of source coils 18, the receiving antenna 310 outputs a magnetic field detection signal corresponding to the intensity of the detected magnetic field to the insertion shape information acquisitor 320.

The insertion shape information acquisitor 320 acquires the position of each of the plurality of source coils 18 based on a magnetic field detection signal output from the receiving antenna 310. Specifically, as the respective positions of the plurality of source coils 18, the insertion shape information acquisitor 320 acquires a plurality of three-dimensional coordinate values in a virtual spatial coordinate system with the origin or reference point at a predetermined position (such as the anus) of the subject. The insertion shape information acquisitor 320 generates insertion shape information including the three-dimensional coordinate values of the plurality of source coils 18, and outputs the insertion shape information to a controller 260 and the external force information acquisition apparatus 40.

The external force information acquisition apparatus 40 acquires the curvature (or radius of curvature) and the bending angle at each position of the plurality of source coils 18 based on the insertion shape information output from the insertion shape detection apparatus 30. The external force information acquisition apparatus 40 may acquire external force information indicating the magnitude and direction of the external force at each position of the plurality of source coils 18 from the acquired curvature (or radius of curvature) and the bending angle and from various data stored in advance. The external force information acquisition apparatus 40 outputs the acquired external force information to the controller 260.

The processing apparatus 20 includes a light source unit 210, a signal processer 220, a coil drive signal generator 230, a driver 240, a display processer 250, and a controller 260. In the embodiment, the processing apparatus 20 serves as an image processing apparatus that processes an endoscopic image. Specifically, the processing apparatus 20 generates information on the movement or operation of the endoscope 10 based on the endoscopic image, and automatically controls the movement of the endoscope 10.

The light source unit 210 generates illumination light for illuminating the inside of the subject and supplies the illumination light to the endoscope 10. The light source unit 210 may have one or more LEDs or one or more lamps as a light source. The light source unit 210 may change the amount of the illumination light in response to a movement control signal supplied from the controller 260.

The signal processer 220 has a signal processing circuit, performs predetermined processing on an imaging signal output from the endoscope 10 so as to generate an endoscopic image, and outputs the generated endoscopic image to the display processer 250 and the controller 260.

The coil drive signal generator 230 generates a coil drive signal for driving the source coil 18. The coil drive signal generator 230 has a drive circuit, generates a coil drive signal in response to a movement control signal supplied from the controller 260, and supplies the coil drive signal to the source coil 18.

The driver 240 generates a control signal corresponding to a basic operation of the endoscope 10 based on a movement control signal supplied from the controller 260, and drives the movement mechanism in the endoscope 10. Specifically, the driver 240 controls at least one of the following movements: an advancement and retraction movement by the advancement and retraction mechanism 141; a bending movement by the bending mechanism 142; an AWS movement by the AWS mechanism 143; and a rotation movement by the rotation mechanism 144. The driver 240 includes an advancement and retraction driver 241, a bending driver 242, an AWS driver 243, and a rotation driver 244.

The advancement and retraction driver 241 generates and outputs an advancement and retraction control signal for controlling the movement of the advancement and retraction mechanism 141 based on the movement control signal supplied from the controller 260. Specifically, the advancement and retraction driver 241 generates and outputs an advancement and retraction control signal for controlling the rotation of a motor provided in the advancement and retraction mechanism 141 based on the movement control signal supplied from the controller 260.

The bending driver 242 generates and outputs a bending control signal for controlling the movement of the bending mechanism 142 based on the movement control signal supplied from the controller 260. Specifically, the bending driver 242 generates and outputs a bending control signal for controlling the rotation of a motor provided in the bending mechanism 142 based on the movement control signal supplied from the controller 260.

The AWS driver 243 generates and outputs an AWS control signal for controlling the movement of the AWS mechanism 143 based on the movement control signal supplied from the controller 260. Specifically, the AWS driver 243 generates and outputs an AWS control signal for controlling the movement state of the solenoid valve provided in the AWS mechanism 143 based on the movement control signal supplied from the controller 260.

The rotation driver 244 generates and outputs a rotation control signal for controlling the movement of the rotation mechanism 144 based on the movement control signal supplied from the controller 260. Specifically, the rotation driver 244 generates and outputs a rotation control signal for controlling the rotation of a motor provided in the rotation mechanism 144 based on the movement control signal supplied from the controller 260.

The display processer 250 generates a display image including an endoscopic image output from the signal processer 220, and displays the generated display image on the display apparatus 60. The display processer 250 may display, on the display apparatus 60, a result image obtained by processing the endoscopic image by the controller 260 and information on the operation detail of the endoscope 10.

The controller 260 according to the embodiment has a function of controlling the driver 240 in either a manual insertion mode in which a physician operates the endoscope 10 or an automatic insertion mode in which the endoscope 10 is automatically operated. When the manual insertion mode of the endoscope 10 is set to ON, the controller 260 has a function of generating a movement control signal for causing the endoscope 10 to perform a movement in accordance with an instruction, etc. from the operation unit 16 and the input apparatus 50 and outputting the movement control signal to the driver 240. Further, when the automatic insertion mode of the endoscope 10 is set to ON, the controller 260 has a function of automatically controlling the movement of the endoscope 10 based on the endoscopic image generated by the signal processer 220. Hereinafter, before explaining the automatic insertion control in the embodiment, manual operation of the endoscope by the physician will be briefly explained.

In the manual insertion mode, the physician views the endoscopic image displayed on the display apparatus 60, confirms a situation around the distal end of the endoscope, and operates the endoscope in accordance with the situation. The physician instantly decides to, for example, avoid an obstacle present near the distal end of the endoscope, avoid contact of the distal end of the endoscope with the mucosal surface, not to place a load to the gastrointestinal tract, and decide on the current path in anticipation of the path ahead, and operates the endoscope.

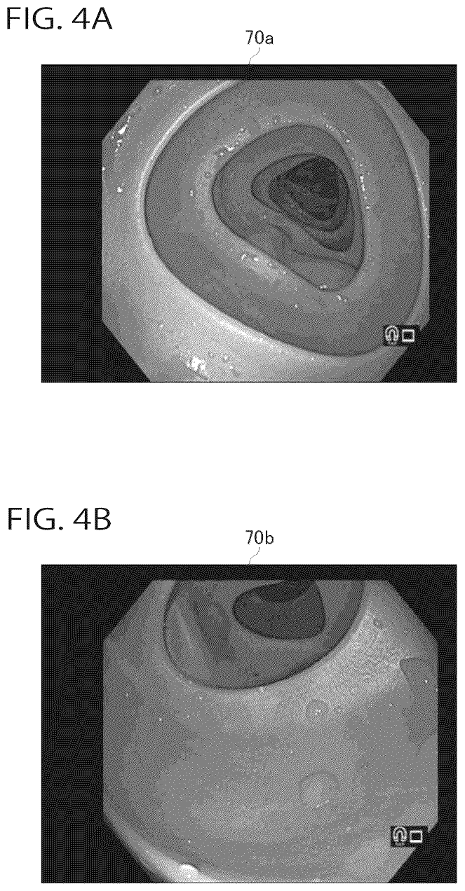

FIG. 4A shows an example of an endoscopic image. An endoscopic image 70a is an image of a colon model including an intestinal tract formed of a material (such as rubber or silicone) imaged by an endoscope. When the physician views the endoscopic image 70a and confirms that the lumen (i.e. the center of the lumen, in other words, a direction in which the center of the lumen exists) exists in the center of the image, the physician decides that the distal end of the endoscope may be advanced and moves the distal end of the endoscope forward.

FIG. 4B shows another example of an endoscopic image. An endoscopic image 70b is also an image obtained by imaging a colon model. The physician views the endoscopic image 70b and confirms that the center of the lumen exists in the upper part of the image, and decides that the distal end of the endoscope will come into contact with a fold in the center of the image if the distal end is moved forward in this state. Thus, the physician operates the angle knob so as to bend the bending portion 13 upward such that the center of the lumen is imaged in the approximate center of the image. When the center of the lumen is imaged in the approximate center of the image, since the state is the same as the state in the endoscopic image 70a shown in FIG. 4A, the physician decides that the distal end of the endoscope may be advanced and moves the distal end of the endoscope forward.

The above decisions and operations can be easily performed only by a physician. Aiming at automation of the movement of the endoscope by an apparatus, it is necessary to correctly recognize the situation around the distal end of the endoscope and identify or estimate the position of the center of the lumen.

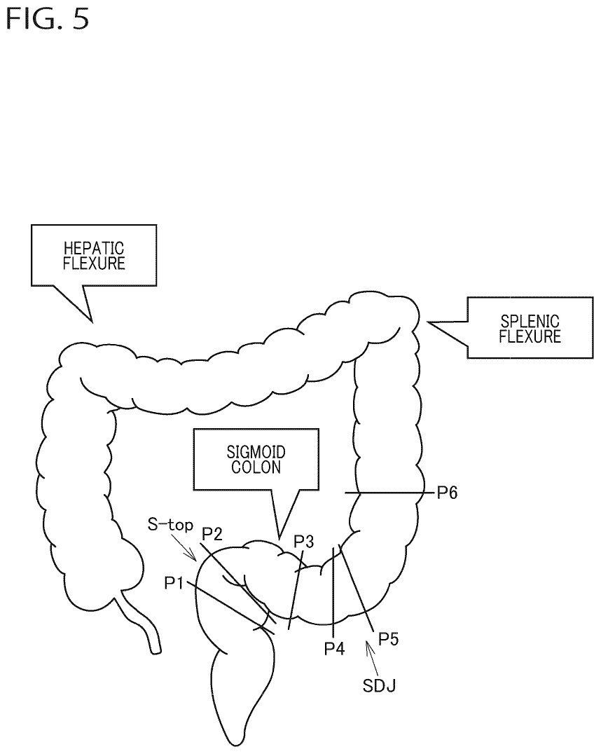

FIG. 5 is a schematic diagram of the large intestine. In the sigmoid colon, a bending portion (a bending portion of the intestinal tract) often appears at the top of the sigmoid colon (commonly referred to as “S-top” by Japanese endoscopists) formed by inserting an endoscope and at the sigmoid-descending colon junction (SDJ). In a subject with a long sigmoid colon, even more abrupt bends may occur. P1 to P6 indicate positions when the distal end of the endoscope passes through the sigmoid colon, and endoscopic images imaged at the respective positions will be explained below.

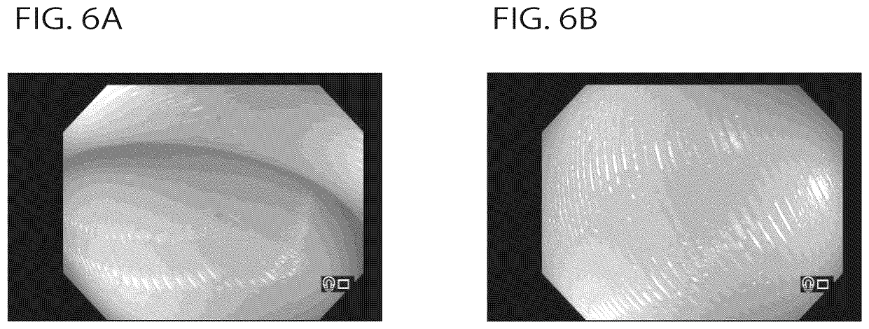

FIG. 6A shows an endoscopic image imaged (captured) when the distal end of the endoscope is located at P1. Since the intestinal tract is bent, two structural components formed by intestinal walls (or folds) and an arc-shaped boundary between the two structural components are imaged in the endoscopic image shown in FIG. 6A. However, the center of the lumen (hereinafter, also simply referred to as “lumen”) is not imaged.

FIG. 6B shows an endoscopic image imaged when the distal end of the endoscope is located at P2. Since the distal end of the endoscope is advanced from P1 to P2 and is close to the intestinal wall, only the intestinal wall is imaged in the endoscopic image shown in FIG. 6B. When the distal end of the endoscope is further advanced from P2, the distal end of the endoscope hits the intestinal wall and causes pain to the subject. Thus, the controller 260 needs to control the movement of the distal end of the endoscope so as not to hit the intestinal wall. The controller 260 according to the embodiment changes the direction of the distal end of the endoscope at P2 to face a direction in which the center of the lumen exists (more accurately, a direction in which the center of the lumen is estimated to exist), and advances the distal end of the endoscope again toward the center of the lumen when the center of the lumen is imaged.

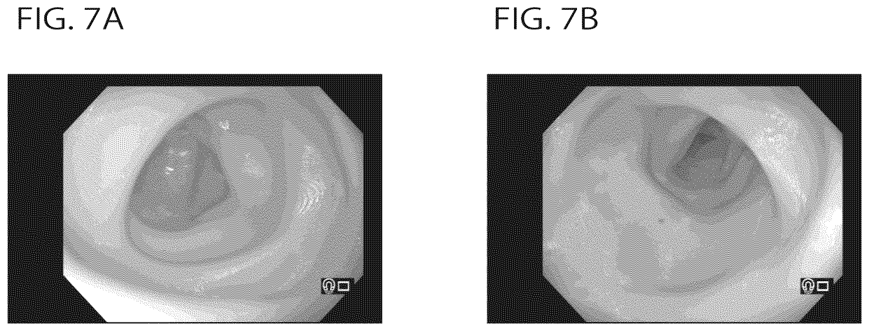

FIG. 7A shows an endoscopic image imaged when the distal end of the endoscope is located at P3. Since the distal end of the endoscope has passed through the top of the sigmoid colon (S-top) as the bending portion, the center of the lumen in the sigmoid colon is clearly imaged in the endoscopic image shown in FIG. 7A. The center of the lumen is imaged in the approximate center of the endoscopic image, and thus the controller 260 may advance the distal end of the endoscope toward the center of the lumen. Furthermore, the controller 260 may deaerate the lumen by appropriately performing the suction movement after the advancement. Not adding too much air into the lumen allows for insertion with less burden on the large intestine. On the back side of the direction of the lumen in FIG. 7A, the sigmoid-descending colon junction (SDJ) as the next bending portion is imaged.

Referring to FIG. 5, when the distal end of the endoscope is advanced from P3 to P4 near the sigmoid-descending colon junction (SDJ), the endoscopic image as shown in FIG. 6A is imaged again. When the distal end of the endoscope is further advanced to P5, the distal end of the endoscope comes close to the intestinal wall, whereby the endoscopic image as shown in FIG. 6B is imaged.

FIG. 7B shows an endoscopic image imaged when the distal end of the endoscope is located at P6. Since the distal end of the endoscope has passed through the sigmoid-descending colon junction (SDJ) as the bending portion, the center of the lumen in the descending colon is clearly imaged in the endoscopic image shown in FIG. 7B. The center of the lumen is imaged in the approximate center of the endoscopic image, and thus the controller 260 may advance the distal end of the endoscope toward the center of the lumen.

As shown in FIGS. 7A and 7B, when the endoscopic image includes the center of the lumen, the controller 260 may control the movement of the endoscope so as to advance toward the center of the lumen. In other words, when the endoscopic image includes the center of the lumen, the controller 260 can control the movement of the endoscope relatively simply. Meanwhile, as shown in FIGS. 6A and 6B, when the endoscopic image does not include the center of the lumen, the controller 260 needs to estimate the position and direction in which the center of the lumen exists and control a series of movements of the endoscope. Even when the endoscopic image includes the center of the lumen, there is a case where it is not preferable to advance the distal end of the endoscope as normal depending on the situation around the distal end of the endoscope. Thus, the controller 260 according to the embodiment has a function of classifying an imaged endoscopic image as any one of a plurality of types, determining a situation around the distal end of the endoscope, and controlling the movement of the endoscope using a control method in accordance with the situation.

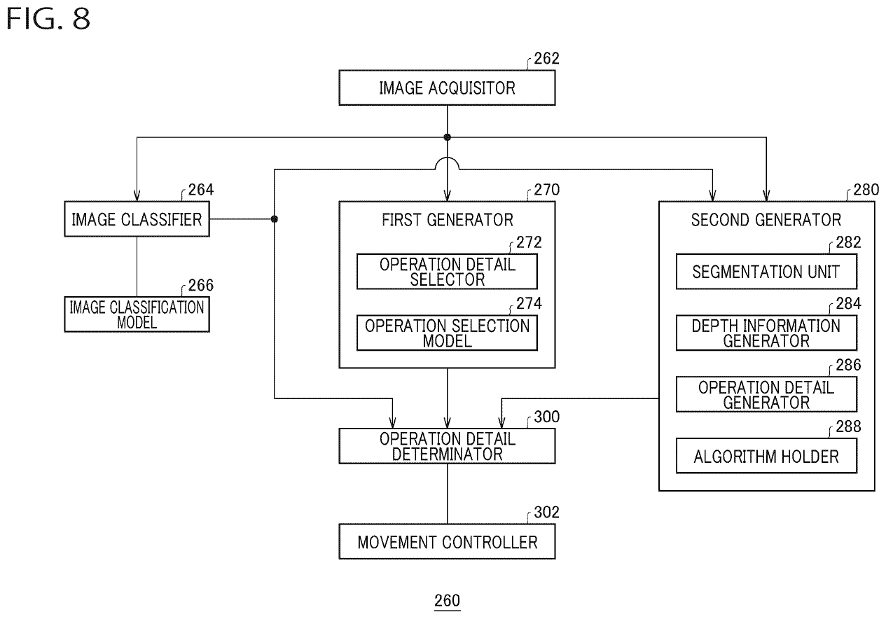

FIG. 8 illustrates functional blocks of the controller 260 according to the embodiment. The controller 260 includes an image acquisitor 262, an image classifier 264, an image classification model 266, a first generator 270, a second generator 280, an operation detail determinator 300, and a movement controller 302. The first generator 270 has an operation detail selector 272 and an operation selection model 274. The second generator 280 has a segmentation unit 282, a depth information generator 284, an operation detail generator 286, and an algorithm holder 288.

The controller 260 illustrated in FIG. 8 includes a computer, and various functions illustrated in FIG. 8 are implemented by the computer executing programs. The computer includes, as hardware, a memory for loading programs, one or more processors for executing the loaded programs, an auxiliary storage device, other LSIs, and the like. The processor includes a plurality of electronic circuits including a semiconductor integrated circuit and an LSI, and the plurality of electronic circuits may be mounted on one chip or may be mounted on a plurality of chips. It should be understood by those skilled in the art that the functional blocks illustrated in FIG. 8 are implemented by cooperation of hardware and software, and thus, these functional blocks can be implemented in various forms by hardware alone, software alone, or a combination thereof.

The image acquisitor 262 acquires an endoscopic image imaged by the endoscope 10 from the signal processer 220. The imager 110 of the endoscope 10 supplies an imaging signal at a predetermined cycle (e.g. 30 frames/second) to the signal processer 220, and the signal processer 220 generates an endoscopic image from the imaging signal and supplies the endoscopic image to the image acquisitor 262. The image acquisitor 262 supplies the acquired endoscopic image to each of the image classifier 264, the first generator 270, and the second generator 280.

First, the movement of the first generator 270 to which the endoscopic image is supplied will be explained.

### Operation Detail Generation Processing by First Generator

The operation detail selector 272 has a function of selecting an operation detail to be performed from a plurality of predetermined operation details based on the endoscopic image acquired by the image acquisitor 262. The plurality of predetermined operation details may include at least one kind of the following operations: an advancement operation; a retraction operation; an angle operation, a twist operation; an air feeding operation; a water feeding operation; and a suction operation.

In response to inputting of input data acquired from the endoscopic image acquired in the image acquisitor 262 to the operation selection model 274, the operation detail selector 272 selects an appropriate operation detail of the endoscope 10 imaging the endoscopic image. The operation selection model 274 is a learned model generated by machine learning using, as training data, an image for learning, which is an endoscopic image imaged in the past, and a label that is assigned to the image for learning and that indicates an operation detail of an endoscope.

In the embodiment, the operation selection model 274 is generated through the learning of each coupling coefficient (weight) in a convolutional neural network (CNN) corresponding to a multilayer neural network including an input layer, one or more convolutional layers, and an output layer by a learning method such as deep learning. The training data to be used includes an image for learning, which is an endoscopic image of the inside of a human intestinal tract or a colon model imaged by an endoscope in the past, and a label indicating which of twelve operation details is most suitable for a situation shown by the image for learning.

The twelve operation details are as follows:
Angle operation UPS for pointing the distal end 12 upward by bending the bending portion 13
Angle operation RIS for pointing the distal end 12 rightward by bending the bending portion 13
Angle operation DOS for pointing the distal end 12 downward by bending the bending portion 13
Angle operation LES for pointing the distal end 12 leftward by bending the bending portion 13
Angle operation URS for pointing the distal end 12 in an upper right direction by bending the bending portion 13
Angle operation DRS for pointing the distal end 12 in a lower right direction by bending the bending portion 13
Angle operation DLS for pointing the distal end 12 in a lower left direction by bending the bending portion 13
Angle operation ULS for pointing the distal end 12 in an upper left direction by bending the bending portion 13
Advancement operation PSS for advancing the distal end 12
Retraction operation PLS for retracting the distal end 12
Search operation SES for pointing the distal end 12 in a plurality of directions to search for the lumen
Angle maintenance operation AMS for maintaining the direction of the distal end 12 to be the current direction by fixing the bending angle of the bending portion 13

The annotation operation of the endoscopic image is performed by an annotator having specialized knowledge such as a physician. The annotator looks at the image for learning, subjectively selects one operation detail that is most likely to be performed in a situation shown in the image for learning from among the twelve operation details described above, and assigns a label of the selected operation detail to the image for learning to create training data.

For example, when the endoscopic image 70a shown in FIG. 4A is an image for learning, since the center of the lumen exists in the approximate center of the image, the annotator determines that the distal end of the endoscope should be advanced, and assigns an advancement operation PSS label to the endoscopic image 70a. Meanwhile, when the endoscopic image 70b shown in FIG. 4B is an image for learning, since the center of the lumen exists in an upper part of the image, the annotator determines that the distal end of the endoscope should be pointed upward, and assigns an angle operation UPS label to the endoscopic image 70b. This label assigning task is performed on a large number of past endoscopic images, thereby generating the training data.

An example of training data including an image for learning and a label is shown below.

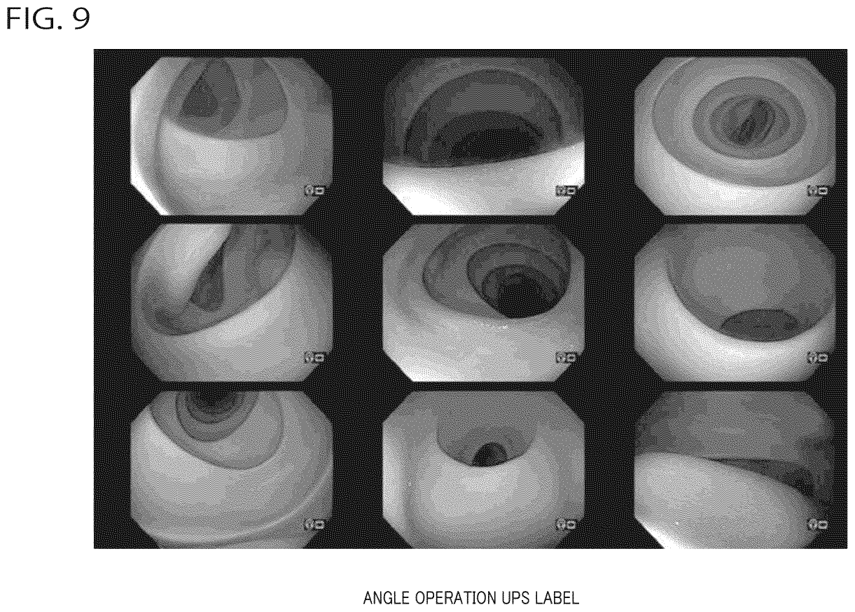

FIG. 9 shows an example of training data. To all images for learning shown in FIG. 9, “angle operation UPS label” indicating an upward angle operation is assigned. The images for learning shown in FIG. 9 are images for which it has been decided that the bending portion 13 should be bent in the upward direction as an endoscope operation to be performed.

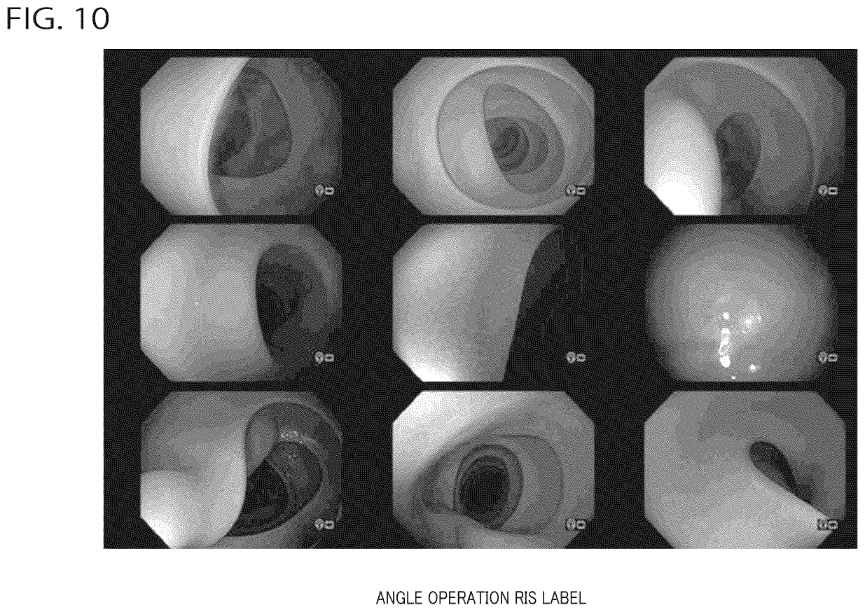

FIG. 10 shows another example of training data. To all images for learning shown in FIG. 10, “angle operation RIS label” indicating a rightward angle operation is assigned. The images for learning shown in FIG. 10 are images for which it has been decided that the bending portion 13 should be bent in the rightward direction as an endoscope operation to be performed.

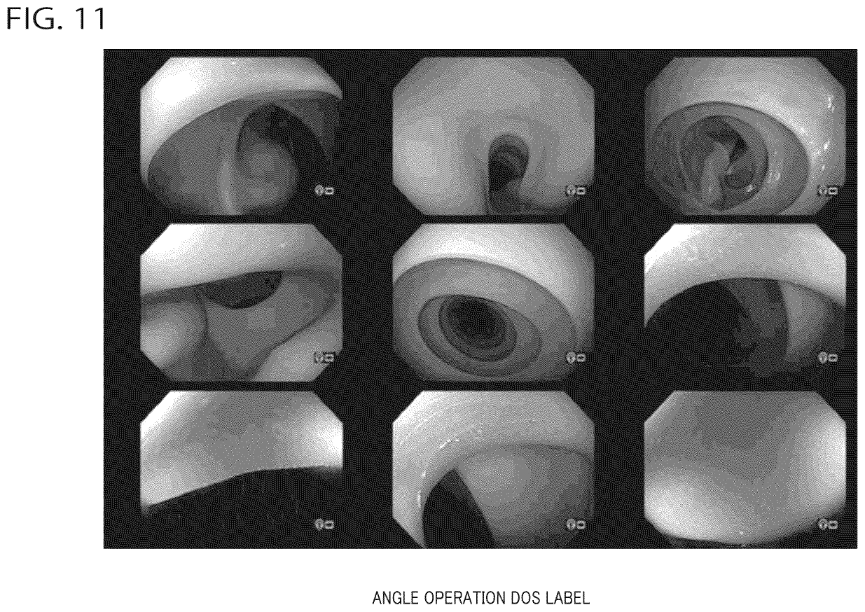

FIG. 11 shows another example of training data. To all images for learning shown in FIG. 11, “angle operation DOS label” indicating a downward angle operation is assigned. The images for learning shown in FIG. 11 are images for which it has been decided that the bending portion 13 should be bent in the downward direction as an endoscope operation to be performed.

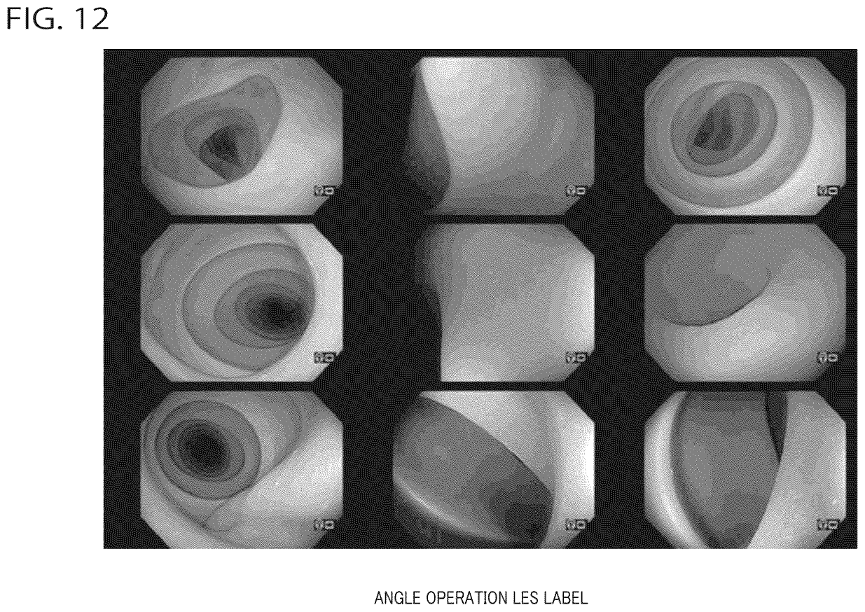

FIG. 12 shows another example of training data. To all images for learning shown in FIG. 12, “angle operation LES label” indicating a leftward angle operation is assigned. The images for learning shown in FIG. 12 are images for which it has been decided that the bending portion 13 should be bent in the leftward direction as an endoscope operation to be performed.

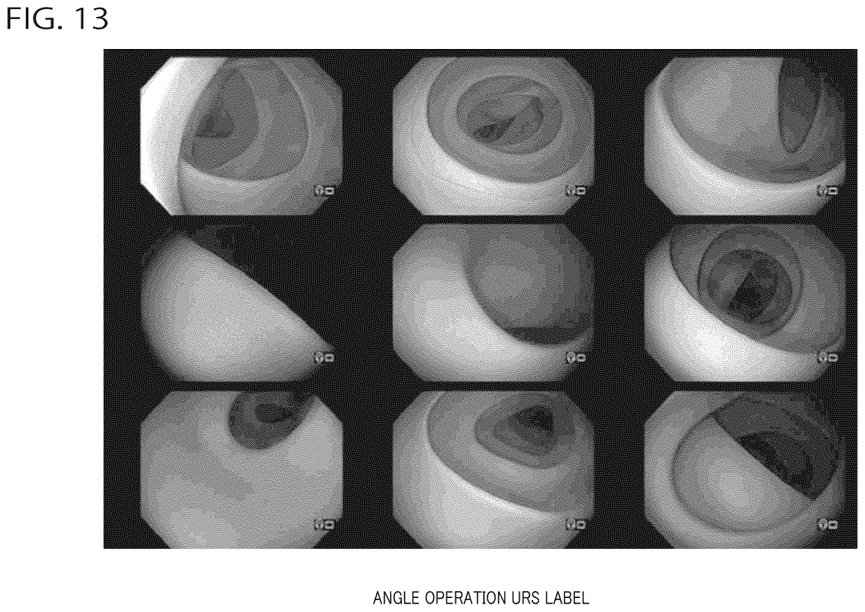

FIG. 13 shows another example of training data. To all images for learning shown in FIG. 13, “angle operation URS label” indicating an angle operation in an upper right direction is assigned. The images for learning shown in FIG. 13 are images for which it has been decided that the bending portion 13 should be bent in an upper right direction as an endoscope operation to be performed.

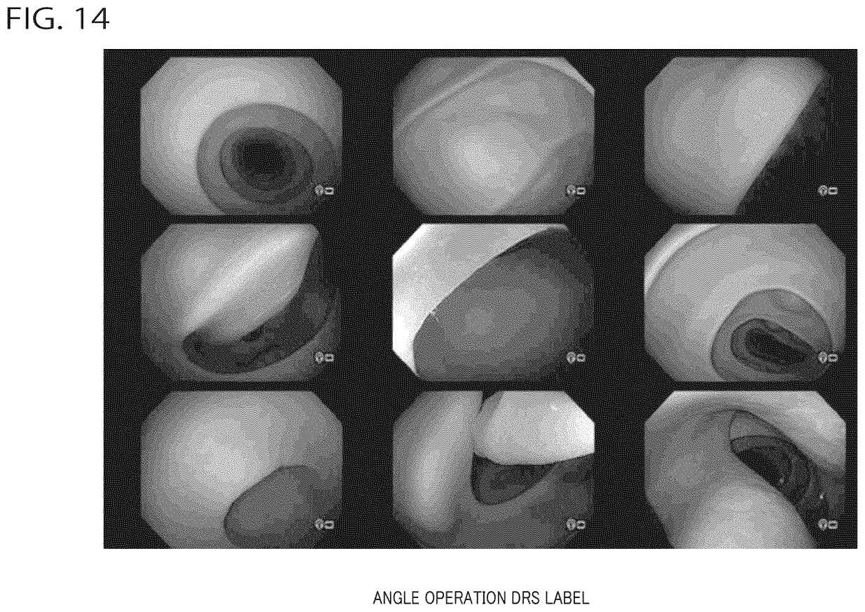

FIG. 14 shows another example of training data. To all images for learning shown in FIG. 14, “angle operation DRS label” indicating an angle operation in a lower right direction is assigned. The images for learning shown in FIG. 14 are images for which it has been decided that the bending portion 13 should be bent in a lower right direction as an endoscope operation to be performed.

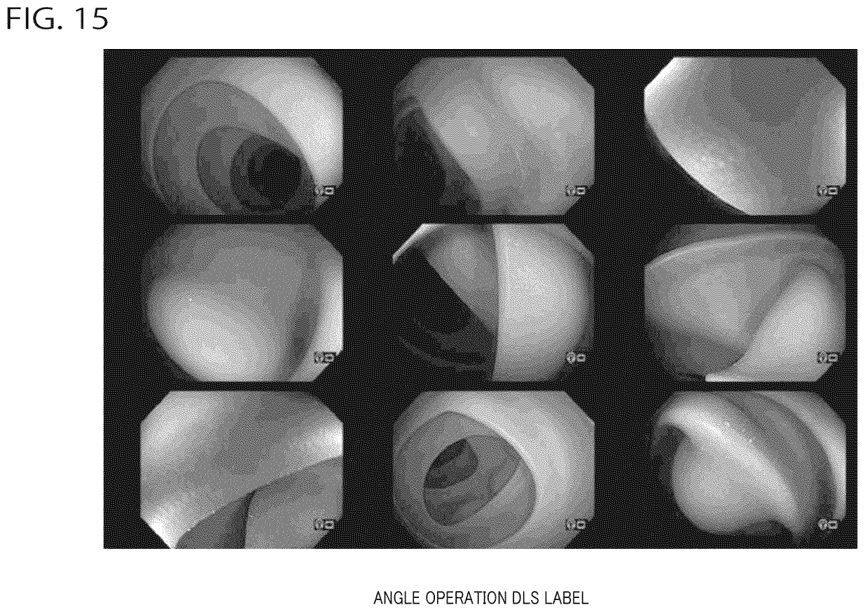

FIG. 15 shows another example of training data. To all images for learning shown in FIG. 15, “angle operation DLS label” indicating an angle operation in a lower left direction is assigned. The images for learning shown in FIG. 15 are images for which it has been decided that the bending portion 13 should be bent in a lower left direction as an endoscope operation to be performed.

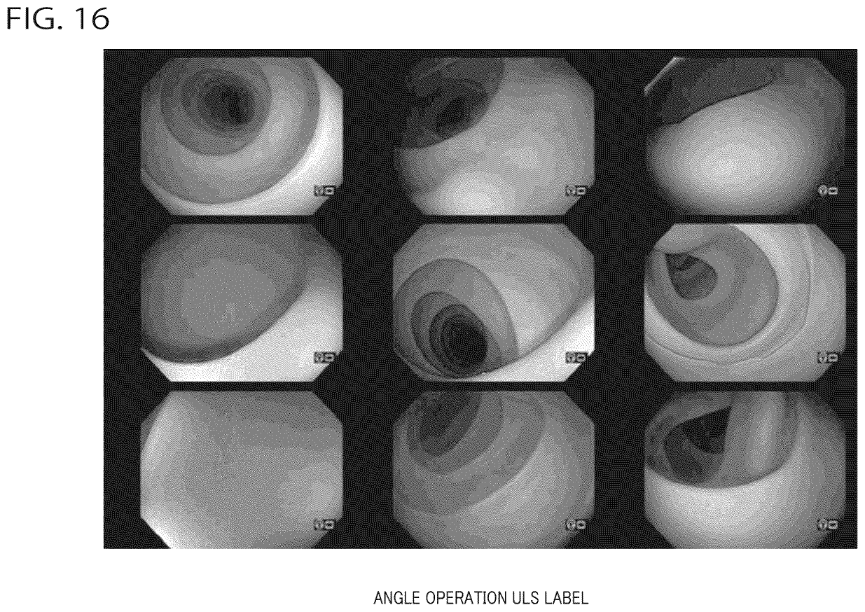

FIG. 16 shows another example of training data. To all images for learning shown in FIG. 16, “angle operation ULS label” indicating an angle operation in an upper left direction is assigned. The images for learning shown in FIG. 16 are images for which it has been decided that the bending portion 13 should be bent in an upper left direction as an endoscope operation to be performed.

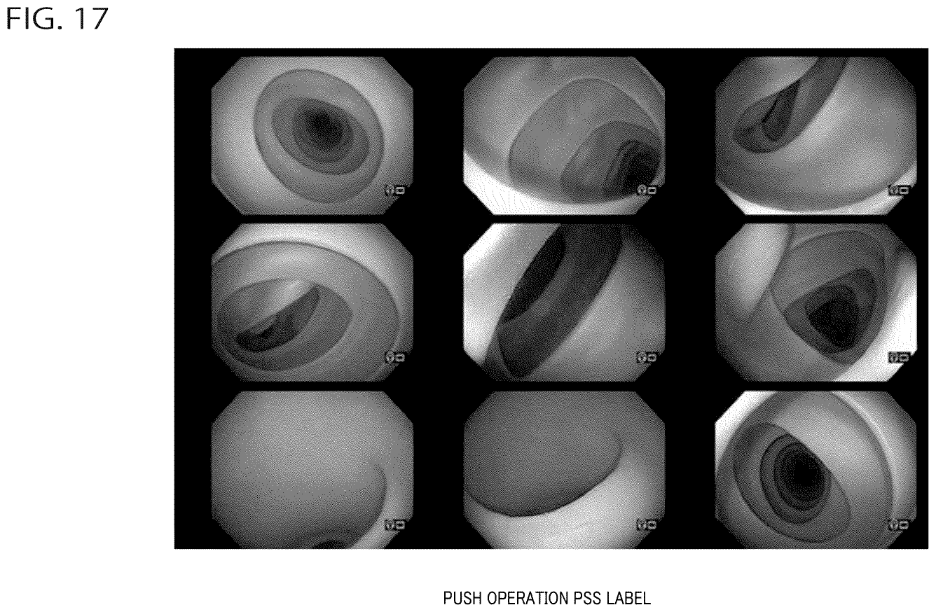

FIG. 17 shows another example of training data. To all images for learning shown in FIG. 17, “push operation (advancement operation) PSS label” indicating an advancement operation is assigned. The images for learning shown in FIG. 17 are images for which it has been decided that the distal end 12 should be advanced as an endoscope operation to be performed.

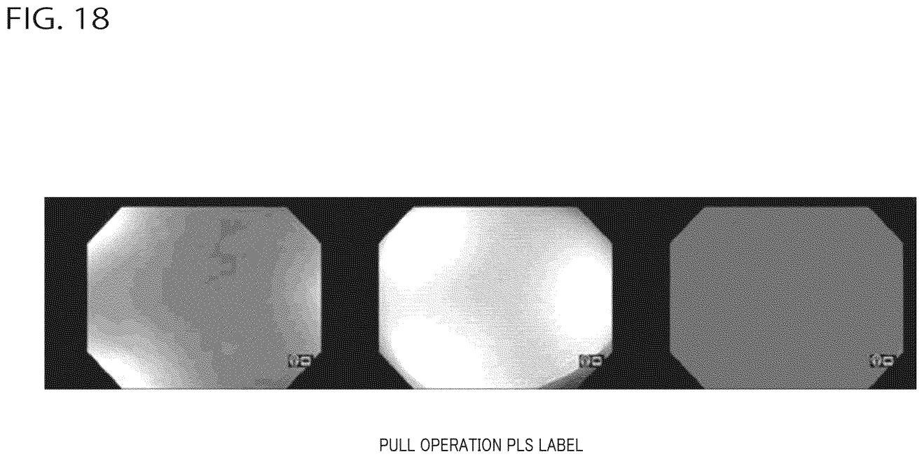

FIG. 18 shows another example of training data. To all images for learning shown in FIG. 18, “pull operation (retraction operation) PLS label” indicating a retraction operation is assigned. The images for learning shown in FIG. 18 are images for which it has been decided that the distal end 12 should be retracted as an endoscope operation to be performed. Here, a typical example of a situation where a retraction operation is required is a situation such as a situation where the distal end 12 is excessively close to the mucosal surface of the large intestine and a situation where the distal end 12 is in contact with the mucosal surface, which is commonly referred to as “red out” by endoscopists.

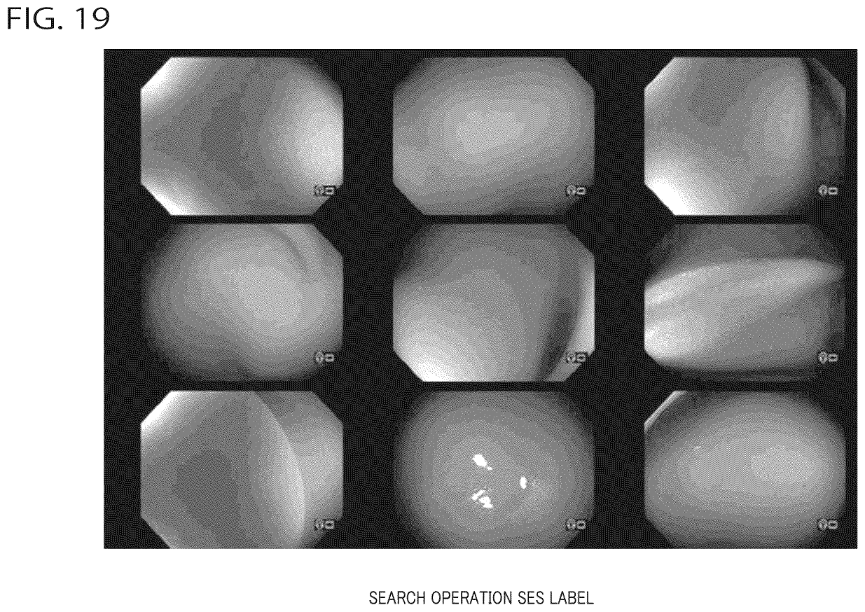

FIG. 19 shows another example of training data. To all images for learning shown in FIG. 19, “search operation SES label” indicating a search operation is assigned. The images for learning shown in FIG. 19 are images for which it has been decided that the bending portion 13 should be bent in a plurality of directions so as to image images in the plurality of directions as an endoscope operation to be performed.

Illustration is omitted for training data of the angle maintenance operation AMS for fixing the bending angle of the bending portion 13 and maintaining the direction of the distal end 12 to be the current direction. However, for example, a label for “angle maintenance operation AMS” may be assigned to an image for learning shown in FIG. 17.

As described above, the operation selection model 274 is generated by machine learning using the training data shown in FIGS. 9 to 19. The bending angle in the operation of bending in eight directions, the moving amount (advancing amount) in the push operation, and the moving amount (retracting amount) in the pull operation are set to predetermined values, respectively. For example, the bending angle may be set to 20 degrees, and the advancing amount and the retracting amount may be set to 20 mm.

The operation detail selector 272 selects one operation detail from among a plurality of options by inputting input data acquired from an imaged endoscopic image to the operation selection model 274. Specifically, the operation detail selector 272 acquires multidimensional data such as the pixel value of each pixel included in an endoscopic image, and inputs the multidimensional data as input data to an input layer of the neural network of the operation selection model 274. The operation selection model 274 outputs twelve likelihoods respectively corresponding to the twelve operation details that can be selected as the operation detail of the endoscope 10 from an output layer of the neural network. The operation detail selector 272 can obtain an operation detail corresponding to one likelihood that is the highest among the twelve likelihoods included in the output data as the result of selecting the operation detail of the endoscope 10.

As described above, the operation detail selector 272 is formed so as to obtain one selection result from among twelve operation details including: operations for changing the direction the distal end 12 at a predetermined angle to eight directions orthogonal to the insertion axis of the insertion portion 11, respectively; an operation for advancing or retracting the distal end 12 at a predetermined distance; an operation for maintaining the direction of the distal end 12 to be the current direction; and an operation for searching for a lumen near the distal end 12, by inputting input data acquired from the endoscopic image into the operation selection model 274 for processing. The operation detail selection processing using the operation selection model 274 has the advantage of being executed in a short time with a small amount of computation. The operation detail selector 272 supplies the selected operation detail to the operation detail determinator 300.

In general, the output accuracy of the operation selection model 274 is improved by learning using a lot of training data. However, for example, in a special situation where the center of the lumen is not imaged or not clearly imaged (which is not a normal situation), the operation detail determined using the operation selection model 274 may not be appropriate.

A scene in which the distal end 12 passes through the bending portion will be explained with reference to FIG. 5. The distal end 12 images an endoscopic image (see FIG. 6A) including the boundary of the bending portion at Pl, then advances to P2 close to the intestinal wall, and images an endoscopic image (see FIG. 6B) including only the intestinal wall. After that, the distal end 12 changes its direction to face a direction in which the center of the lumen exists (a direction in which the center of the lumen is estimated to exist), and advances to the center of the lumen when the center of the lumen is imaged. In order for the distal end 12 to pass through the bending portion as described above, it is necessary to perform a series of movements of advancing to a position close to the intestinal wall while capturing the boundary of the arc-shaped folds in the field of view, changing the direction at the position to search for the center of the lumen, and advancing to the center of the lumen so as to get out of the bending portion.

However, it is not easy to execute this series of movements using the operation selection model 274. For example, when the endoscopic image shown in FIG. 6B is imaged, the operation selection model 274 selects the pull operation (retraction operation) PLS to avoid a collision between the distal end and the intestinal wall, and a situation where the distal end 12 retracts may occur. At this time, the distal end 12 may repeat the movement of once retreating and then advancing again, or retreating and then advancing.

It is conceivable to prepare many class labels for the purpose of coping with various situations and cause the operation selection model 274 to learn. However, it is necessary to prepare an enormous number of training data corresponding to each of the situations, and it is practically difficult. Accordingly, the endoscope control system 1 according to the embodiment recognizes the current situation of the distal end 12 of the endoscope 10 from the endoscopic image acquired by the image acquisitor 262, determines the operation detail using the operation selection model 274 when the situation allows, and determines the operation detail using another method by the second generator 280 when the situation does not allow.

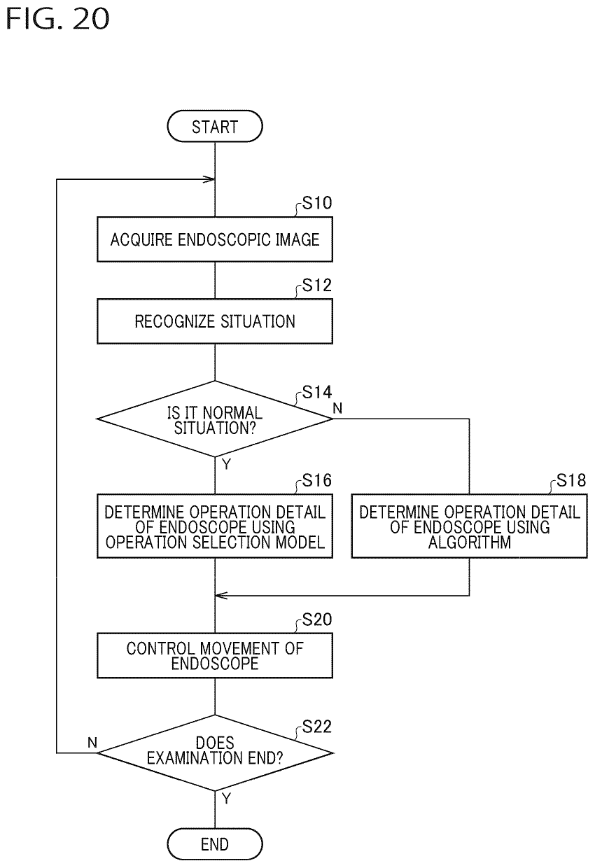

FIG. 20 illustrates a flowchart for determining operation details of the endoscope. When the image acquisitor 262 acquires an endoscopic image (S10), the image classifier 264 classifies the endoscopic image as any one of a plurality of types and recognizes a situation around the distal end 12 (S12).

The image classifier 264 identifies the type of the endoscopic image by inputting input data acquired from the endoscopic image to the image classification model 266. The image classification model 266 is a learned model generated by machine learning using, as training data, an image for learning, which is an endoscopic image imaged in the past, and a label indicating a type of the image for learning. In the embodiment, the image classification model 266 is generated through the learning of each coupling coefficient (weight) in a convolutional neural network (CNN) corresponding to a multilayer neural network including an input layer, one or more convolutional layers, and an output layer by a learning method such as deep learning.

In the embodiment, four types of class labels are used.
First type: a normal image (an image mainly including a lumen)
Second type: an image of a bending portion (an image including a bending portion)
Third type: an image of a stenosis part (an image including a stenosis part)
Fourth type: an image of diverticulum (an image including a diverticulum)

The first type of endoscopic image includes a clearly imaged lumen and is suitable for processing of determining an operation detail using the operation selection model 274. An endoscopic image including a structural component (obstacle) that interferes with the passing of the distal end of the endoscope, even when the endoscopic image includes a clearly imaged lumen, may be classified as another type (e.g. the third type or the fourth type) instead of the first type.

An endoscopic image that is not classified as the first type does not include a clearly imaged lumen and/or includes a structural component that interferes with the passing of the distal end of the endoscope, and is not suitable for the processing of determining an operation detail using the operation selection model 274. For example, the second type of endoscopic image may include at least an image which includes a boundary of a bending portion and does not include a clear lumen, the third type of endoscopic image may include at least an image which includes a very narrowed lumen due to stenosis, and the fourth type of endoscopic image may include at least an image which includes a lumen and one or more diverticula.

The annotator looks at an image for learning, identifies a type of the image for learning, assign a label of the identified type to the image for learning, and creates training data. A first type label is assigned to the images for learning shown in FIGS. 7A and 7B, and a second type label is assigned to the image for learning shown in FIG. 6A.

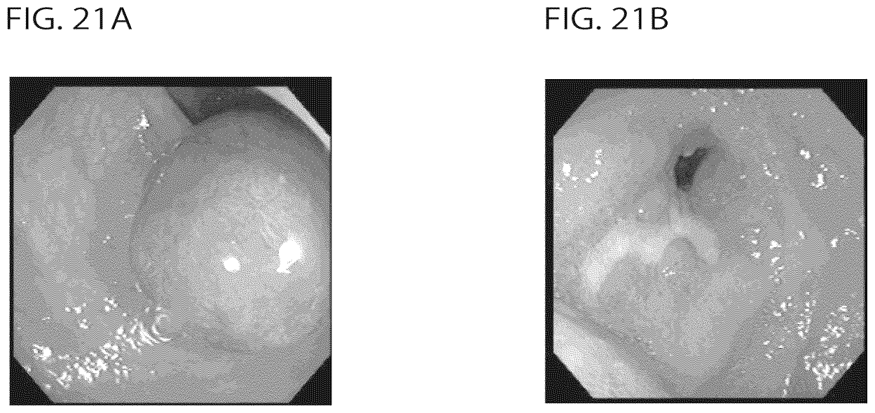

FIGS. 21A and 21B show examples of images for learning to which a third type label is assigned. In the embodiment, the term “stenosis part” refers to a lumen site that has narrowed for some reason. FIG. 21A shows an image obtained by imaging a stenosis part caused by a large lesion, and FIG. 21B shows an image obtained by imaging a stenosis part caused by an influence of surgical operation or an influence of inflammation. Both the images show a very narrow lumen. The advancing amount of the advancing movement determined using the operation selection model 274 is fixed to 20 mm, but it is preferable to carefully perform the advancement with an advancing amount smaller than 20 mm upon passing through the stenosis part.

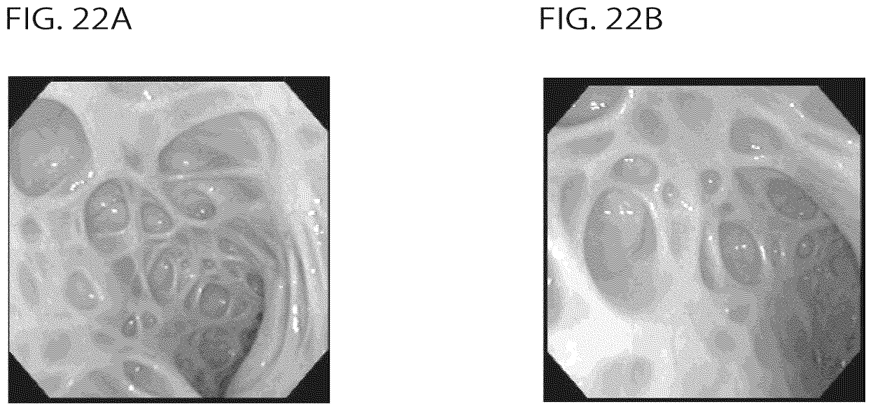

FIGS. 22A and 22B show examples of images for learning to which a fourth type label is assigned. FIGS. 22A and 22B both show multiple diverticula. When a human physician looks at such an endoscopic image, the physician can distinguish between the lumen and the diverticula. However, when the neural network is used, it is difficult to distinguish between the lumen and the diverticula. Thus, there is a possibility that the lumen and the diverticula are erroneously determined or erroneously extracted. In this situation, it is not easy to determine the operation detail using the operation selection model 274 because there is a plurality of lumen candidates and it is not clear which lumen candidate is the true lumen.

The image classification model 266 is generated by machine learning using training data. The image classifier 264 identifies the type of the endoscopic image by inputting input data acquired from the endoscopic image to the image classification model 266. Specifically, when the image classifier 264 inputs multidimensional data such as the pixel value of each pixel included in an endoscopic image to an input layer of the neural network of the image classification model 266, the image classification model 266 outputs four likelihoods respectively corresponding to four types that can be selected as image types from an output layer of the neural network. The image classifier 264 acquires an image type corresponding to one likelihood that is the highest among the four likelihoods included in the output data as the type of the endoscopic image.

As described above, the image classifier 264 classifies the endoscopic image as any one of the four types using the image classification model 266, and recognizes a situation around the distal end 12. When the endoscopic image is classified as the first type, the image classifier 264 recognizes that the situation around the distal end 12 is a normal situation (Y in S14), and notifies the operation detail determinator 300 and the second generator 280 of the recognition.

Upon receiving this notification, the operation detail determinator 300 determines the operation detail selected by the first generator 270 as the operation detail of the endoscope 10 (S16), and the movement controller 302 controls the movement of the endoscope 10 in accordance with the operation detail determined using the operation selection model 274 (S20). When the endoscopic image is thus classified as the first type, the operation detail determinator 300 determines the operation detail of the endoscope 10 using the operation selection model 274. The operation detail selection processing using the operation selection model 274 has the advantage of being executed in a short time with a small amount of computation. Thus, the stability of the automatic insertion control of the endoscope 10 is enhanced.

Meanwhile, when the endoscopic image is classified as a type different from the first type, the image classifier 264 recognizes that the situation around the distal end 12 is a special situation (N in S14), and notifies the second generator 280 and the operation detail determinator 300 of the recognition.

Upon receiving this notification, the second generator 280 generates an operation detail of the endoscope 10 using an algorithm suitable for the situation around the distal end 12, and the operation detail determinator 300 determines the operation detail generated by the second generator 280 as the operation detail of the endoscope 10 (S18). An algorithm for determining the operation detail is prepared for each situation around the distal end 12, and the second generator 280 generates a series of operation details of the endoscope 10 using the algorithm suitable for the situation around the distal end 12. The movement controller 302 controls the movement of the endoscope 10 in accordance with the operation detail determined using the algorithm (S20). Even when a special situation is recognized, the first generator 270 may continue to supply the operation detail selected using the operation selection model 274 to the operation detail determinator 300, but the operation detail determinator 300 may discard the information provided from the first generator 270.

During the examination (N in S22), when the movement control in S20 is ended, the process returns to the step of S10 and continues. When the examination is ended (Y in S22), the movement control of the endoscope is ended.

Hereinafter, the movement of the second generator 280 will be explained.

### Operation Detail Generation Processing by Second Generator

The second generator 280 has a function of extracting various structural components, such as lumen and folds, from an endoscopic image using an image recognition method of performing structural image analysis, and generating an appropriate operation detail based on the size and positional relationship. The algorithm holder 288 holds a program for realizing an algorithm for determining the operation detail for each situation around the distal end of the endoscope. In the embodiment, the algorithm holder 288 holds a program for realizing a bending portion passage algorithm, a program for realizing a stenosis part passage algorithm, and a program for realizing a diverticulum passage algorithm. The operation detail generator 286 generates a series of operation detail of the endoscope using an algorithm suitable for the situation of the distal end of the endoscope based on the information on the structural component included in the endoscopic image.

When an endoscopic image imaged by the endoscope 10 is acquired, the image acquisitor 262 supplies the endoscopic image to the second generator 280. In the second generator 280, the endoscopic image is processed by the segmentation unit 282 and the depth information generator 284.

### Segmentation Processing of Endoscopic Image

The segmentation unit 282 has a function of partitioning the endoscopic image acquired by the image acquisitor 262 into a plurality of regions. Specifically, the segmentation unit 282 executes semantic segmentation of labeling each pixel in the endoscopic image to partition the endoscopic image into regions corresponding to a plurality of predetermined structures. The segmentation unit 282 defines a region having a structure of a type (a class) to be partitioned, and generates a segmentation result obtained by labeling pixels of various structures. Semantic segmentation is realized using a fully convolutional neural network (FCN), a bilateral segmentation network (BiSeNet), or the like, but the segmentation unit 282 may execute semantic segmentation using the FCN.

As the type (the class) of the region to be partitioned, label values up to 0 to 255 may be prepared. In the embodiment, label values are assigned to the following structures:
Label value 0: background pixel
Label value 1: lumen
Label value 2: fold edge (contour)
Label value 3: edge of bending portion

In semantic segmentation, the label value 0 generally means “a region not to be extracted”, whereas the label value 0 defined in the embodiment means the mucosal surface. The “lumen” to which the label value 1 is assigned means a structure in which the endoscope can be advanced in the endoscopic image, and is defined as a structure indicating an advancing direction of the distal end of the endoscope. The structure specifically defined as “lumen” represents an extending direction of the lumen. Note that, in addition to these classes, classes may be set for structures such as residues, polyps, and blood vessels that appear in large intestine endoscopy, and label values may be assigned to these classes, respectively.

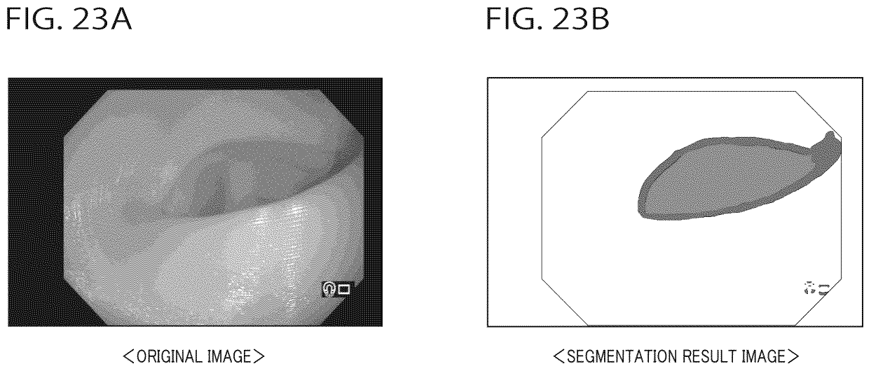

FIG. 23A shows an example of an endoscopic image. The endoscopic image has a size of 720×480, and each pixel of RGB is represented by eight bits. In this endoscopic image, the lumen extending in the depth direction is imaged, and folds surround the lumen.

FIG. 23B shows an example of a segmentation result by the segmentation unit 282. The segmentation unit 282 partitions the endoscopic image into a plurality of regions and derives region information indicating a result of segmentation. The region information is derived as a label value pa (x, y) of each pixel related to the structure, and the segmentation unit 282 generates a segmentation result image using the derived label value. The segmentation result image may be displayed on the display apparatus 60 and presented to the user.

The segmentation unit 282 may set the pixel value of (R, G, B) corresponding to the label value of the partitioned region as follows. In the following description, in order to distinguish it from the label value related to the depth information, the label values 0 to 3 of the partitioned region are expressed as label values a0 to a3.
Label value a0 (background pixel)→(255,255,255)
Label value a1 (lumen)→(128,0,0)
Label value a2 (fold edge)→(0,0,128)
Label value a3 (edge of bending portion)→(128,128,128)

With the pixel values set in this manner, the segmentation unit 282 generates a segmentation result image in which the mucosal surface occupying a large part (label value a0) is painted in white and the extracted structural part is painted in color. The segmentation unit 282 supplies the segmentation result image to the operation detail generator 286 as region information indicating a result of segmentation. In the example shown in FIG. 23B, a fold edge region and a lumen region surrounded by the fold edge are displayed so as to be visible to the user. In still another example, the segmentation unit 282 may supply the label value of each pixel to the operation detail generator 286 as region information indicating a result of segmentation.

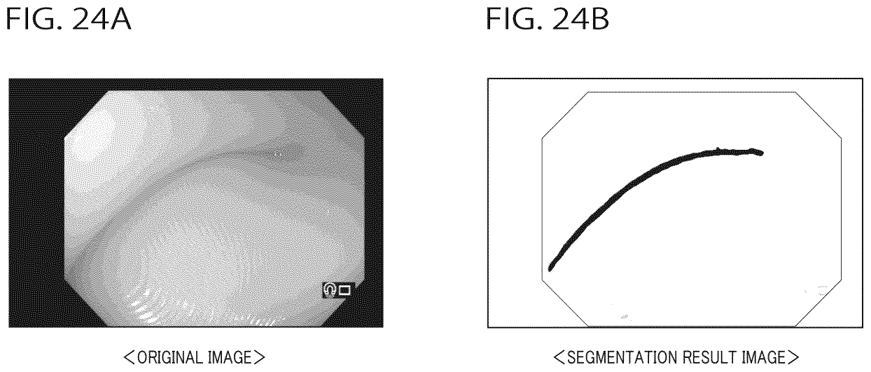

FIG. 24A shows another example of an endoscopic image. In this endoscopic image, the lumen is not imaged, and the boundary of the bending portion is imaged.

FIG. 24B shows an example of a segmentation result by the segmentation unit 282. The segmentation result image includes a region of an edge (boundary) of the bending portion extending in the lateral direction near the center. The segmentation unit 282 supplies the segmentation result image to the operation detail generator 286 as region information indicating a result of segmentation.

### Depth Information Generation Processing of Endoscopic Image

The depth information generator 284 has a function of generating information indicating the depth of the endoscopic image acquired by the image acquisitor 262. In the related art, various methods for estimating the depth of a pixel or a block included in an image have been proposed. Faisal Mahmood, Richard Chen, Nicholas J. Durr, “Unsupervised Reverse Domain Adaptation for Synthetic Medical Images via Adversarial Training”, 29 Nov. 2017 <URL: https://arxiv.org/pdf/1711.06606.pdf> uses three-dimensional information by CT colonography as training data of distance information, but the depth information generator 284 may generate information indicating the depth of each pixel of the endoscopic image using the technique disclosed in Faisal Mahmood, Richard Chen, Nicholas J. Durr, “Unsupervised Reverse Domain Adaptation for Synthetic Medical Images via Adversarial Training”, 29 Nov. 2017 <URL: https://arxiv.org/pdf/1711.06606.pdf>.

Note that the depth information generator 284 may generate a learning model for the depth estimation processing based on training data that has been simply created. For example, a creator (annotator) of the training data may create the training data by visually designating each stage of the label values 0 to 4 in accordance with a positional relationship in a depth direction for each region of the image. In this case, a relative positional relationship in the depth direction based on human senses is obtained. Although it is not easy to obtain distance information as an absolute numerical value from a normal endoscopic image, it is easy for a person skilled in viewing an endoscopic image to determine that it is near or distant view. In addition, and the physician actually performs an insertion operation using the sensuous distance information obtained from the image. Therefore, the training data created in this manner has high reliability, making it possible to generate a learning model capable of estimating an accurate depth.

In a depth estimation method by the depth information generator 284, a class is set in accordance with a distance range from the distal end 12 of the endoscope. In the embodiment, a label value is assigned to each distance range.
Label value 0: depth<first distance
Label value 1: first distance≤depth<second distance
Label value 2: second distance≤depth<third distance
Label value 3: third distance≤depth<fourth distance
Label value 4: fourth distance≤depth

The label value 0 indicates a region having the shortest distance from the distal end 12, and the label value 4 indicates a region having the farthest distance from the distal end 12.

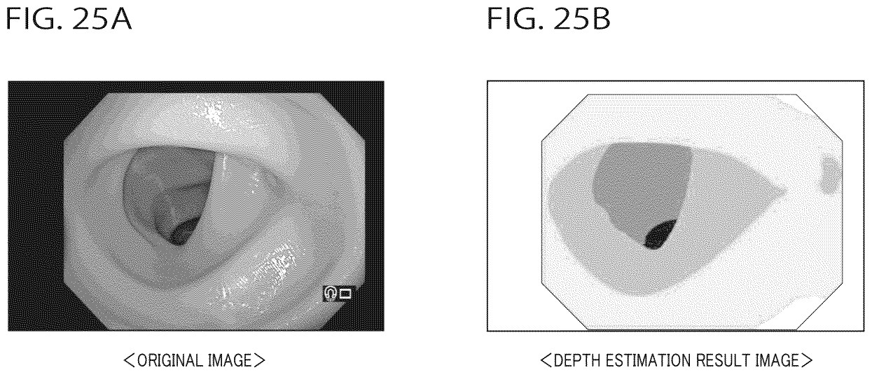

FIG. 25A shows another example of an endoscopic image. In this endoscopic image, the lumen extending in the depth direction is imaged.

FIG. 25B shows an example of a depth information estimation result by the depth information generator 284. The depth information generator 284 executes the depth estimation processing on the endoscopic image to generate depth information indicating the depth of the endoscopic image. Here, the depth information may be derived as a label value of each pixel related to the depth (distance from the distal end of the endoscope). In the embodiment, the depth information generator 284 generates a depth estimation result image using the derived label value. The depth estimation result image may be displayed on the display apparatus 60 together with the segmentation result image and presented to the user.

The depth information generator 284 may set the pixel value of (R,G,B) corresponding to the label value that represents a level of the depth as follows. In the following description, the label values 0 to 4 of the depth information are expressed as label values d0 to d4 in order to distinguish them from the label value related to the partitioned region.
Label value d0 (less than first distance)→(200,0,0)
Label value d1 (first distance or more and less than second distance)→(160,0,0)
Label value d2 (second distance or more and less than third distance)→(120,0,0)
Label value d3 (third distance or more and less than fourth distance)→(80,0,0)
Label value d4 (fourth distance or more)→(40,0,0)

With the pixel values set in this manner, the depth information generator 284 generates a depth estimation result image in which a deeper region is colored in dark red and a closer region is colored in bright red. The depth information generator 284 supplies the depth estimation result image to the operation detail generator 286 as the depth information of the endoscopic image. In still another example, the depth information generator 284 may supply the label value of each pixel to the operation detail generator 286 as the depth information of the endoscopic image.

### Method of Passing Distal End of Endoscope Through Bending Portion

Hereinafter, a method of passing a distal end of an endoscope through a bending portion will be explained using an algorithm.

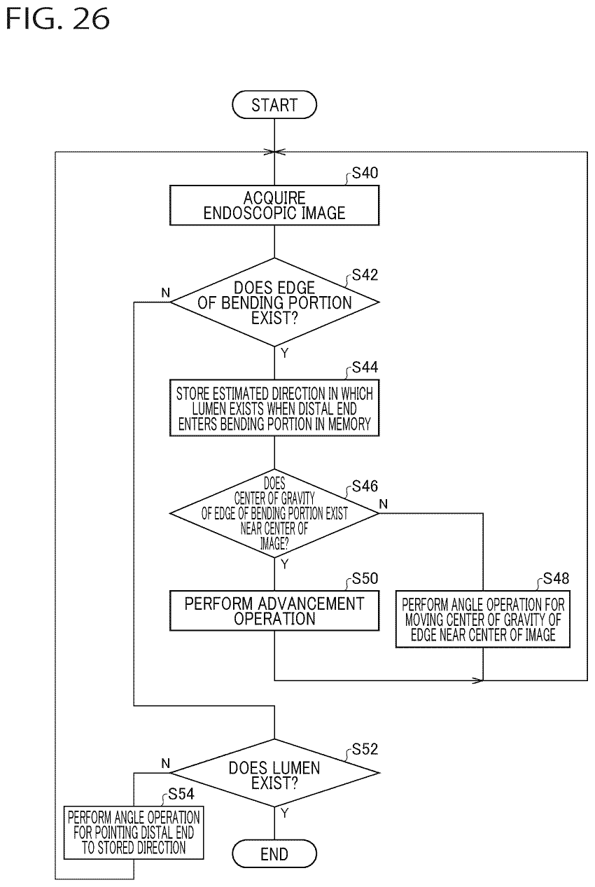

FIG. 26 is a flowchart illustrating a procedure in which the distal end of the endoscope passes through a bending portion. When the image classifier 264 classifies the imaged endoscopic image as the second type, the second generator 280 is activated, and the operation detail generator 286 reads the program for realizing the bending portion passage algorithm from the algorithm holder 288 and starts processing of generating a series of operation details for passing the distal end of the endoscope through the bending portion.

The second generator 280 receives the second type of the endoscopic image including the boundary of the bending portion from the image acquisitor 262 (S40).

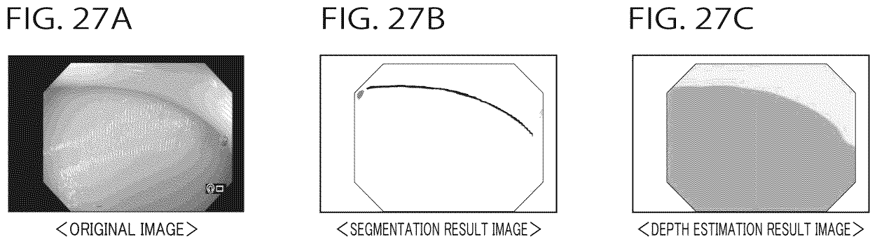

FIG. 27A shows the second type of the endoscopic image including the boundary of the bending portion. The segmentation unit 282 partitions the endoscopic image into a plurality of regions, generates region information indicating a result of segmentation, and provides the region information to the operation detail generator 286. FIG. 27B shows the segmentation result image generated by the segmentation unit 282. Further, the depth information generator 284 executes the depth estimation processing on the endoscopic image to generate depth information indicating the depth of the endoscopic image, and provides the depth information to the operation detail generator 286. FIG. 27C shows the depth estimation result image generated by the depth information generator 284. The operation detail generator 286 recognizes that the edge of the bending portion exists from the segmentation result image shown in FIG. 27B (Y in S42), and recognizes that the structural component on the upper side of the image exists on the front side and the structural component on the lower side of the image exists on the back side from the depth estimation result image shown in FIG. 27C.

The bending portion is formed by overlapping two structural components (folds or intestinal walls) in the depth direction. Thus, there is usually a high possibility that the lumen extending in a direction from the structural component on the back side toward the structural component on the front side exists at a hidden position behind the structural component on the front side. For example, in the bending portion shown in FIG. 27A, the structural component on the front side is included in the upper part of the image, and the structural component on the back side is included in the lower part of the image. Thus, there is a high possibility that the lumen extending in a direction from the structural component on the lower side of the image toward the structural component on the upper side of the image exists at the back of the structural component on the upper side of the image. The operation detail generator 286 uses this estimation theory to estimate that the lumen exists on the upper side based on the region information and the depth information, and stores the estimated direction in which the lumen exists in a memory (not illustrated) (S44).

Subsequently, the operation detail generator 286 determines whether or not the center of gravity of the edge of the bending portion exists near the center of the image (S46). The presence of the center of gravity of the edge of the bending portion near the center of the image is a condition for advancing the distal end of the endoscope. The operation detail generator 286 determines whether or not the center of gravity of a group of pixels extracted as the edge of the bending portion exists near the center of the image from the region information.

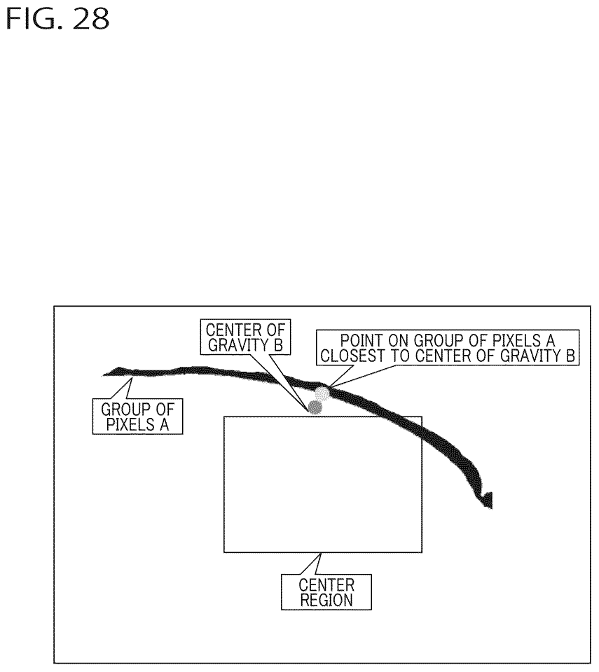

FIG. 28 is a diagram for explaining a method of obtaining a center of gravity B of a group of pixels A extracted as the edge of the bending portion. In a case where the center of gravity B is located on the group of pixels A, the operation detail generator 286 uses the position of the center of gravity B as it is as a position of the center of gravity. Meanwhile, in a case where the center of gravity B is not located on the group of pixels A but is located at a position deviated from the group of pixels A, the operation detail generator 286 may use a position on the group of pixels A closest to the center of gravity B as the position of the center of gravity. The operation detail generator 286 determines that the center of gravity of the edge of the bending portion exists near the center of the image when the position of the center of gravity is included in the “center region”, whereas determines that the center of gravity of the edge of the bending portion does not exist near the center of the image when the position of the center of gravity is not included in the “center region”.

The operation detail generator 286 obtains the position of the center of gravity of the edge of the bending portion from the segmentation result image shown in FIG. 27B and determines that the position of the center of gravity of the edge of the bending portion does not exist near the center of the image (N in S46). At this time, in order to move the center of gravity of the edge near the center of the image, the operation detail generator 286 generates an operation detail for pointing the distal end in the upward direction and notifies the operation detail determinator 300 of the operation detail. The operation detail determinator 300 determines the upward angle operation generated by the operation detail generator 286 as an operation detail to be performed (S48). The movement controller 302 generates a movement control signal corresponding to the determined operation detail and supplies the movement control signal to the driver 240. In this case, the movement controller 302 supplies a movement control signal for bending the angle upward to the driver 240, and the driver 240 bends the bending portion 13 upward and changes the direction of the distal end 12 upward. For example, the bending angle may be 10 degrees.

After changing the direction of the distal end 12 to an upward direction, the second generator 280 receives the endoscopic image from the image acquisitor 262 (S40).

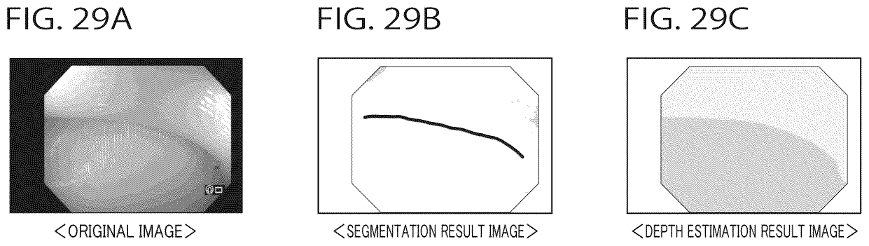

FIG. 29A shows the endoscopic image imaged after changing the direction of the distal end 12. The segmentation unit 282 generates region information indicating a result of segmentation, and provides the region information to the operation detail generator 286. FIG. 29B shows the segmentation result image generated by the segmentation unit 282. Further, the depth information generator 284 generates depth information indicating the depth of the endoscopic image and provides the depth information to the operation detail generator 286. FIG. 29C shows the depth estimation result image generated by the depth information generator 284. The operation detail generator 286 determines that an edge of a bending portion exists from the segmentation result image shown in FIG. 29B (Y in S42). Further, the operation detail generator 286 uses the estimation theory to estimate that the lumen exists on the upper side based on the region information and the depth information, and stores the estimated direction in which the lumen exists in a memory (not illustrated) (S44).

Subsequently, the operation detail generator 286 obtains the position of the center of gravity of the edge of the bending portion from the segmentation result image shown in FIG. 29B and determines that the position of the center of gravity of the edge of the bending portion exists near the center of the image (Y in S46). Since the presence of the center of gravity of the edge of the bending portion near the center of the image is a condition for advancing the distal end of the endoscope, the operation detail generator 286 generates an operation detail to be advanced and notifies the operation detail determinator 300 of the operation detail. The operation detail determinator 300 determines the advancement operation generated by the operation detail generator 286 as the operation detail to be performed (S50), the movement controller 302 supplies a movement control signal for advancing the distal end 12 to the driver 240, and the driver 240 advances the distal end 12. For example, the advancing amount may be set to 20 mm.

After advancing the distal end 12, the second generator 280 receives the endoscopic image from the image acquisitor 262 (S40)

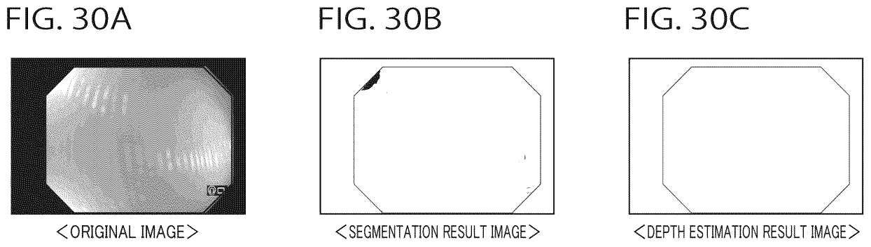

FIG. 30A shows the endoscopic image imaged after advancing the distal end 12. The segmentation unit 282 generates region information indicating a result of segmentation, and provides the region information to the operation detail generator 286. FIG. 30B shows the segmentation result image generated by the segmentation unit 282. Further, the depth information generator 284 generates depth information indicating the depth of the endoscopic image and provides the depth information to the operation detail generator 286. FIG. 30C shows the depth estimation result image generated by the depth information generator 284.

The endoscopic image shown in FIG. 30A is an image imaged in proximity to the intestinal tract, similarly to the endoscopic image shown in FIG. 6B. That is, as the distal end 12 advances in S50, the distal end 12 enters the bending portion. The operation detail generator 286 determines that the edge of the bending portion does not exist from the segmentation result image shown in FIG. 30B (N in S42).

The operation detail generator 286 determines whether or not the lumen exists from the segmentation result image shown in FIG. 30B (S52). Since no lumen is included in the segmentation result image shown in FIG. 30B, the operation detail generator 286 determines that no lumen exists (N in S52). At this time, the operation detail generator 286 generates an operation detail for pointing the distal end in a direction stored in the memory in S44, and notifies the operation detail determinator 300 of the operation detail. In this example, since “upward direction” is stored in the memory, the operation detail generator 286 notifies the operation detail determinator 300 of the operation detail of pointing the distal end in the upward direction. The operation detail determinator 300 determines the upward angle operation generated by the operation detail generator 286 as an operation detail to be performed (S54). The movement controller 302 generates a movement control signal corresponding to the determined operation detail and supplies the movement control signal to the driver 240. In this case, the movement controller 302 supplies a movement control signal for bending the angle upward to the driver 240, and the driver 240 bends the bending portion 13 upward and changes the direction of the distal end 12 upward. For example, the bending angle may be 20 degrees.

After changing the direction of the distal end 12 to an upward direction, the second generator 280 receives the endoscopic image from the image acquisitor 262 (S40).

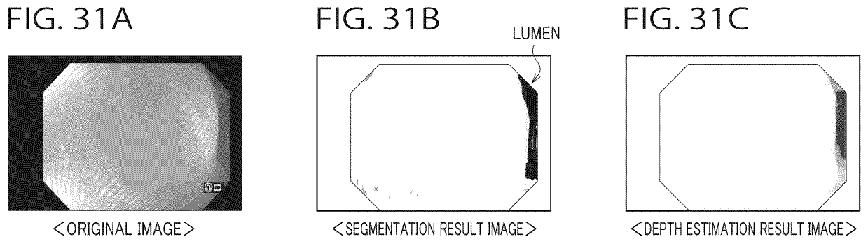

FIG. 31A shows the endoscopic image imaged after changing the direction of the distal end 12 to an upward direction. The segmentation unit 282 generates region information indicating a result of segmentation, and provides the region information to the operation detail generator 286. FIG. 31B shows the segmentation result image generated by the segmentation unit 282. Further, the depth information generator 284 generates depth information indicating the depth of the endoscopic image and provides the depth information to the operation detail generator 286. FIG. 31C shows the depth estimation result image generated by the depth information generator 284. The operation detail generator 286 determines that the edge of the bending portion does not exist from the segmentation result image shown in FIG. 31B (N in S42).

The lumen is included on the right side of the segmentation result image shown in FIG. 31B, and the operation detail generator 286 determines that the lumen exists (Y in S52). When the lumen is included in the endoscopic image, the operation detail generator 286 ends the operation detail generation processing using the bending portion passage algorithm and notifies the operation detail determinator 300 of the end of the processing. At this time, the image classifier 264 classifies the endoscopic image shown in FIG. 31A as the first type, and notifies the operation detail determinator 300 and the second generator 280 that the situation around the distal end 12 is a normal situation. Upon receiving this notification, the operation detail generator 286 may end the operation detail generation processing. After receiving the notification, the operation detail determinator 300 determines not the operation detail generated by the second generator 280 but the operation detail selected by the first generator 270 as the operation detail of the endoscope 10. The movement controller 302 controls the movement of the endoscope 10 in accordance with the operation detail determined using the operation selection model 274.

As described above, when the bending portion is included in the endoscopic image, the second generator 280 performs the operation detail generation processing using the bending portion passage algorithm, and thus a series of operation details suitable for passing the distal end of the endoscope through the bending portion can be determined, which contributes to shortening of the examination time.

Note that there is also a situation where it is difficult for the distal end 12 to smoothly pass through the bending portion due to the reason that the sigmoid colon is excessively long, or the like. In such a situation, it is preferable to avoid forcible continuation of the insertion operation. For example, when the angle operation in the same direction continues a predetermined number of times (e.g. 7 times) successively or when the advancement operation continues a predetermined number of times (e.g. 5 times) successively, it may be determined that the distal end 12 fails to pass through the bending portion and the distal end 12 may be retracted once. After the retraction, the operation detail generator 286 may restart the operation detail generation processing using the bending portion passage algorithm again.

In the flowchart illustrated in FIG. 26, with regard to the estimation processing of the direction in which the lumen exists (S44), it is also possible to use the CNN learned using the training data in which the estimation direction of the lumen is set as the class with respect to the image of the bending portion.

### Method of Passing Distal End of Endoscope Through Stenosis Part

Hereinafter, a method of passing a distal end of an endoscope through a stenosis part using an algorithm will be explained.

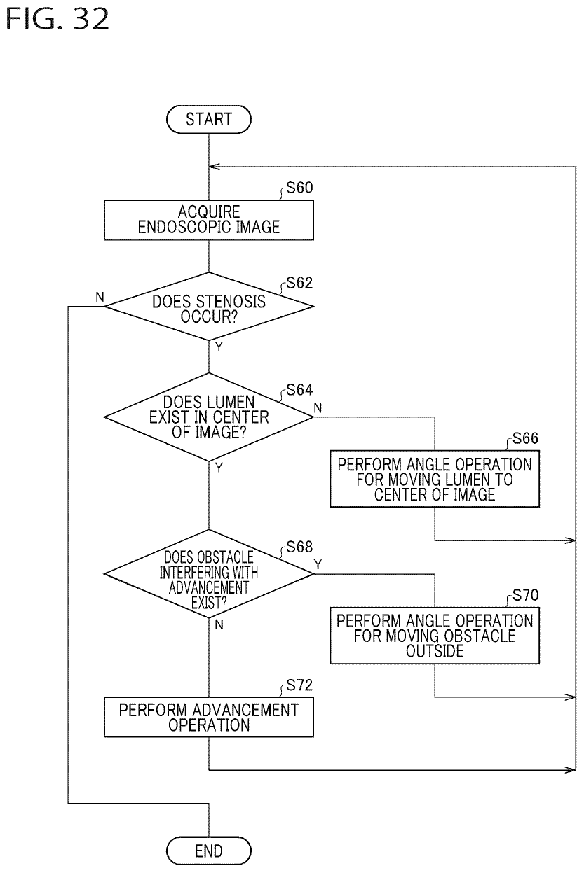

FIG. 32 is a flowchart illustrating a procedure in which the distal end of the endoscope passes through the stenosis part. When the lumen is blocked by a lesion or the like, or when the lumen itself is narrowly deformed due to an influence of surgical operation or an influence of inflammation, stenosis occurs. As the movement of passing the endoscope through the stenosis part, it is desirable that the distal end 12 is accurately directed to the stenosed lumen and is carefully inserted into the lumen while avoiding contact with the lesion or the like.

When the image classifier 264 classifies the imaged endoscopic image as the third type, the second generator 280 is activated, and the operation detail generator 286 reads the program for realizing the stenosis part passage algorithm from the algorithm holder 288 and starts processing of generating a series of operation details for passing the distal end of the endoscope through the stenosis part.

The second generator 280 receives the third type of the endoscopic image including the stenosis part from the image acquisitor 262 (S60). The third type of the endoscopic image includes the stenosis part (Y in S62).

The operation detail generator 286 determines whether or not the center of gravity of the lumen exists in the center of the image (S64). The operation detail generator 286 determines whether or not the center of gravity of the group of pixels extracted as the lumen exists in the center of the image from the region information generated by the segmentation unit 282.

When the center of gravity of the lumen does not exist in the center of the image (N in S64), the operation detail generator 286 generates an operation detail for changing the direction of the distal end in order to move the center of gravity of the lumen to the center of the image, and notifies the operation detail determinator 300 of the operation detail. The operation detail determinator 300 determines the angle operation generated by the operation detail generator 286 as an operation detail to be performed (S66). The movement controller 302 generates a movement control signal corresponding to the determined operation detail and supplies the movement control signal to the driver 240. The driver 240 bends the bending portion 13 in accordance with the movement control signal to change the direction of the distal end 12. For example, the bending angle may be 10 degrees.

After changing the direction of the distal end 12, the second generator 280 receives the endoscopic image including the stenosis part (Y in S60 and S62). When the center of gravity of the lumen exists in the center of the image in the endoscopic image (Y in S64), the operation detail generator 286 determines whether or not a structural component (fold or intestinal wall) interfering with the advancement of the distal end 12 exists around the lumen, from the depth information generated by the depth information generator 284 (S68).

When a structural component (obstacle) interfering with the advancement exists (Y in S68), the operation detail generator 286 generates an operation detail for changing the direction of the distal end in order to move the obstacle in the direction toward the outside of the image (move the obstacle away from the lumen) while maintaining the position of the lumen in the center of the image, and notifies the operation detail determinator 300 of the operation detail. The operation detail determinator 300 determines the angle operation generated by the operation detail generator 286 as an operation detail to be performed (S70). The movement controller 302 generates a movement control signal corresponding to the determined operation detail and supplies the movement control signal to the driver 240. The driver 240 bends the bending portion 13 in accordance with the movement control signal to change the direction of the distal end 12.

When no obstacle exists (N in S68), the operation detail generator 286 generates an operation detail to be advanced and notifies the operation detail determinator 300 of the operation detail. The operation detail determinator 300 determines the advancement operation generated by the operation detail generator 286 as an operation detail to be performed (S72), the movement controller 302 supplies a movement control signal for advancing the distal end 12 to the driver 240, and the driver 240 advances the distal end 12. For example, it is preferable that the advancing amount is set to 10 mm or 5 mm and the distal end 12 is carefully advanced.

After advancing the distal end 12, when a lumen without stenosis is included in the endoscopic image (N in S62), the operation detail generator 286 ends the operation detail generation processing using the stenosis part passage algorithm, and notifies the operation detail determinator 300 of the end of the processing. At this time, the image classifier 264 classifies the endoscopic image acquired by the image acquisitor 262 as the first type, and notifies the operation detail determinator 300 and the second generator 280 that the situation around the distal end 12 is a normal situation. Upon receiving this notification, the operation detail generator 286 may end the operation detail generation processing. After receiving the notification, the operation detail determinator 300 determines not the operation detail generated by the second generator 280 but the operation detail selected by the first generator 270 as the operation detail of the endoscope 10. The movement controller 302 controls the movement of the endoscope 10 in accordance with the operation detail determined using the operation selection model 274.

As described above, when the stenosis part is included in the endoscopic image, the second generator 280 performs the operation detail generation processing using the stenosis part passage algorithm, and thus a series of operation details suitable for passing the distal end of the endoscope through the stenosis part can be determined, which contributes to shortening of the examination time.

### Method of Passing Distal End of Endoscope Through Multiple Diverticulum

Hereinafter, a method of passing a distal end of an endoscope through the multiple diverticulum using an algorithm will be explained.

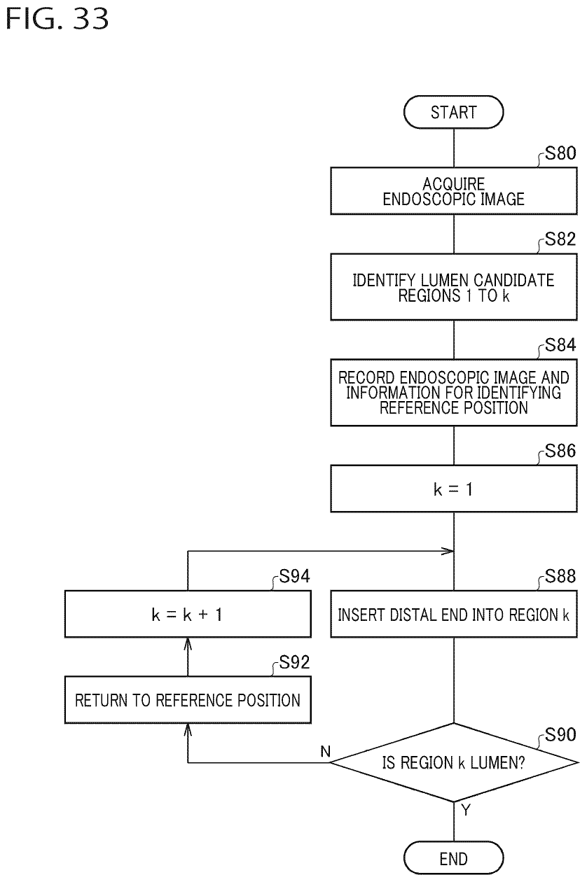

FIG. 33 is a flowchart illustrating a procedure in which the distal end of the endoscope passes through the multiple diverticulum. The diverticulum may show a structure in which the intestinal wall partially protrudes outside the large intestine and which is similar to the lumen. As the movement of the endoscope when one or more diverticula are present in the endoscopic image, it is desirable that a true lumen is searched from a plurality of lumen candidate regions, and the distal end 12 passes through the true lumen.

When the image classifier 264 classifies the imaged endoscopic image as the fourth type, the second generator 280 is activated, and the operation detail generator 286 reads the program for realizing the diverticulum passage algorithm from the algorithm holder 288 and starts processing of generating a series of operation details for passing the distal end of the endoscope through the diverticulum. When a plurality of lumen candidate regions is extracted from the endoscopic image, the diverticulum passage algorithm sets a reference position of the distal end 12, causes the distal end 12 to slightly enter each region from the reference position, and generates a series of operation details for investigating whether or not the region is a true lumen.

The second generator 280 receives the fourth type of the endoscopic image including one or more diverticula from the image acquisitor 262 (S80).

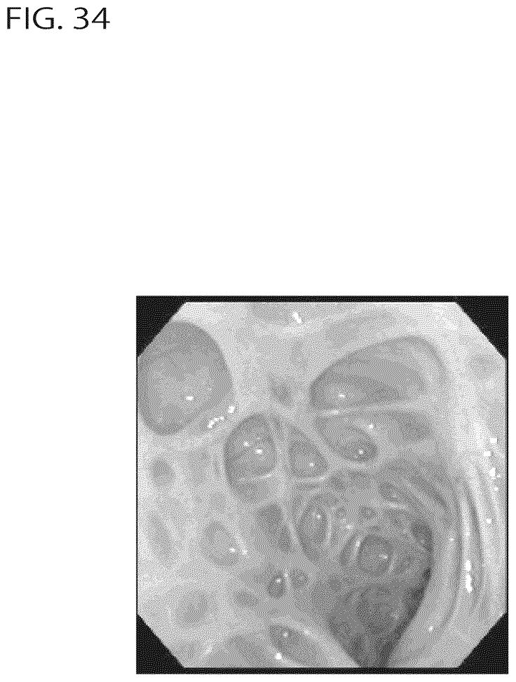

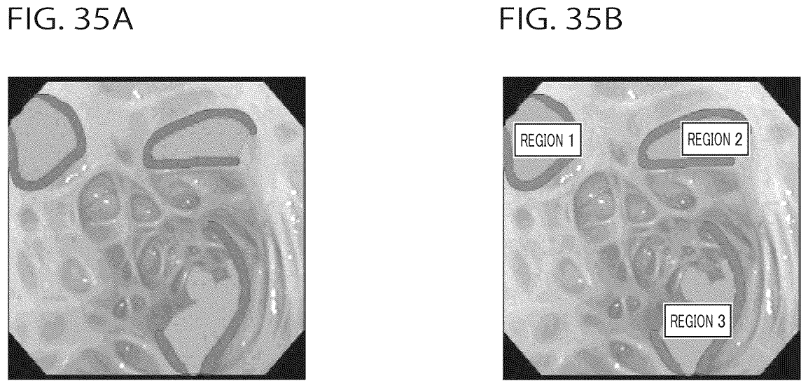

FIG. 34 shows the fourth type of the endoscopic image including one or more diverticula. The segmentation unit 282 partitions the endoscopic image into a plurality of regions, generates region information indicating a result of segmentation, and provides the region information to the operation detail generator 286. FIG. 35A shows an image in which the lumen region and the fold edge region extracted by the segmentation unit 282 are superimposed on the endoscopic image shown in FIG. 34. In the image shown in FIG. 35A, three lumen regions are extracted, which means that the segmentation unit 282 has erroneously extracted at least two diverticula as the lumen. Therefore, the operation detail generator 286 generates a series of operation details for searching for a true lumen region from a plurality of lumen regions.

The operation detail generator 286 assigns numbers to the lumen regions derived by the segmentation unit 282 and handles them as lumen candidate regions 1 to k (S82). In this example, k=3. FIG. 35B shows a state in which region numbers are assigned to the lumen candidate regions.

In this algorithm, the distal end 12 is controlled to enter each of the lumen candidate regions 1 to k from the reference position to confirm whether or not the candidate is a true lumen. When it is determined that the candidate is not a lumen as a result of the entry, the distal end 12 returns to the reference position and enters another lumen candidate region to confirm whether or not the candidate is a true lumen. Therefore, the operation detail generator 286 records the endoscopic image shown in FIG. 34 and information (reference position information) for identifying the reference position when the endoscopic image is imaged in a memory (not illustrated) (S84). The reference position information may be information for identifying the state of the endoscope 10 when the distal end 12 is at the reference position, and may be, for example, an insertion length and an insertion shape of the endoscope 10.

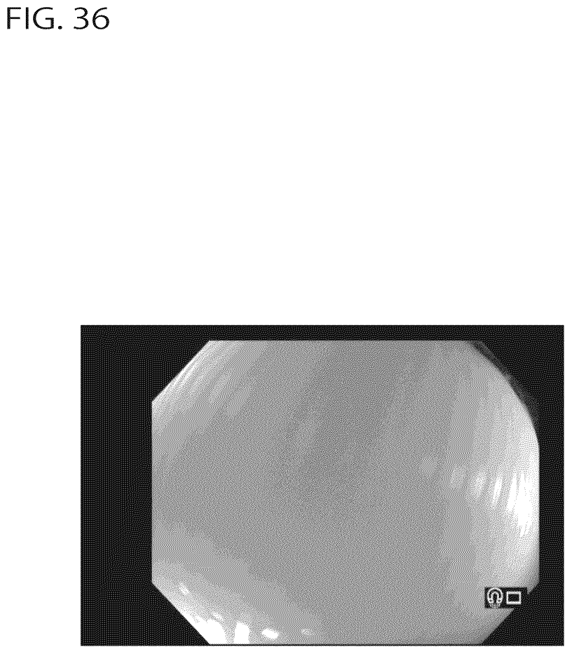

Hereinafter, the movement of confirming the lumen candidate region will be explained. When k is set to 1 (S86), and the operation detail generator 286 sequentially generates an operation for inserting the distal end 12 into the region 1 and notifies the operation detail determinator 300 of the operation. The operation detail determinator 300 determines the operation generated by the operation detail generator 286 as an operation detail to be performed. The movement controller 302 generates a movement control signal corresponding to the determined operation detail and supplies the movement control signal to the driver 240. When the distal end 12 reaches the region 1, the driver 240 advances by a predetermined advancing amount. The advancing amount is set to, for example, 5 mm. When the region 1 is not a true lumen, the distal end 12 is carefully advanced so as not to contact the mucosal surface (S88). When the region 1 is a diverticulum, an endoscopic image as shown in FIG. 36, for example, is imaged by the distal end 12 being close to the mucosal surface. When an image close to the mucosal surface is imaged, the operation detail generator 286 determines that the passage is a dead end and determines that the region 1 is not the lumen (N in S90).

Subsequently, the operation detail generator 286 sequentially generates an operation for returning the distal end 12 to the reference position based on the information recorded in S84, notifies the operation detail determinator 300 of the operation, and moves the distal end 12 to the reference position. The operation detail generator 286 increments k by one (S94), sequentially generates an operation for inserting the distal end 12 into the region 2, and notifies the operation detail determinator 300 of the operation. When the region 2 is not the lumen (N in S90), the operation detail generator 286 generates the same operation detail for the region 3 and determines whether or not the region 3 is the lumen (S90). When the region 3 is the lumen (Y in S90), the algorithm ends. When no lumen is found as a result of investigation of all the lumen candidate regions, this algorithm also ends.

As described above, when the diverticulum is included in the endoscopic image, the second generator 280 performs the processing of generating the operation detail using the diverticulum passage algorithm, and thus it is possible to determine a series of operation details suitable for passing the distal end of the endoscope through the diverticulum, which contributes to shortening of the examination time.

The present disclosure has been described above based on the embodiments. It is to be understood by those skilled in the art that these embodiments are illustrative, that various modifications can be made to combinations of the components and the processing processes, and that such modifications also fall within the scope of the present disclosure. In the embodiments, the processing when the endoscope 10 is inserted into the large intestine has been described, but the endoscope 10 may be inserted into another organ or may be inserted into a pipe or the like. Note that, in the embodiments, it has been described that the operation detail of the endoscope is determined using the operation selection model generated by machine learning with the image classified as the first type. However, it is also possible to control the determination operation such that the lumen is extracted by, for example, semantic segmentation with respect to the image classified as the first type, and the angle operation is performed in that direction.

In the embodiments, an example has been described in which the operation detail of the endoscope 10 is determined by processing the endoscopic image and automatic insertion control is applied. In a modification, the display processer 250 may display, on the display apparatus 60, information on the operation detail determined by the operation detail determinator 300 as guide information when the physician manually operates the endoscope 10. Further, the display processer 250 may display, on the display apparatus 60, the segmentation result image generated by the segmentation unit 282 and the depth estimation result image generated by the depth information generator 284 as the guide information. The display processer 250 displays, on the display apparatus 60, the information on the operation detail, the segmentation result image, and/or the depth estimation result image, thereby supporting the physician to safely operate the endoscope. The operation detail determined by the operation detail determinator 300 may be recorded as log information.

What is claimed is:

1. An endoscope control system that determines an insertion operation detail of an endoscope, comprising one or more processors having hardware,
wherein the one or more processors are configured to:
acquire an image of a lumen imaged by the endoscope;
classify the acquired lumen image as any one of a plurality of types;
determine the insertion operation detail of the endoscope using an insertion operation selection model generated by machine learning when the lumen image is classified as a first type; and
determine the insertion operation detail of the endoscope using an algorithm for determining the insertion operation detail when the lumen image is classified as a second type.

2. The endoscope control system according to claim 1, wherein the one or more processors are configured to:
control a movement of the endoscope in accordance with the insertion operation detail determined using the insertion operation selection model when the lumen image is of the first type; and
control the movement of the endoscope in accordance with the insertion operation detail determined using the algorithm when the lumen image is of the second type.

3. The endoscope control system according to claim 1, wherein the one or more processors are configured to:
display, on a display apparatus, information on the insertion operation detail determined using the insertion operation selection model when the lumen image is of the first type; and
display, on the display apparatus, information on the insertion operation detail determined using the algorithm when the lumen image is of the second type.

4. The endoscope control system according to claim 1, wherein the one or more processors are configured to classify the lumen image as the second type when the lumen image includes a boundary of a bending portion.

5. The endoscope control system according to claim 1, wherein the one or more processors are configured to classify the lumen image as the second type when the lumen image includes a stenosis part.

6. The endoscope control system according to claim 1, wherein the one or more processors are configured to classify the lumen image as the second type when the lumen image includes a diverticulum.

7. The endoscope control system according to claim 1, wherein the insertion operation selection model is generated by machine learning using, as training data, an image for learning, which is a lumen image imaged in the past, and a label that is assigned to the image for learning and indicates an insertion operation detail of an endoscope.

8. The endoscope control system according to claim 1, wherein the one or more processors are configured to determine the insertion operation detail of the endoscope using the algorithm based on information on a structural component in the lumen image when the lumen image is classified as the second type.

9. The endoscope control system according to claim 8, wherein the one or more processors are configured to determine the insertion operation detail of the endoscope using the algorithm based on information indicating a depth of the lumen image.

10. The endoscope control system according to claim 1, wherein the one or more processors are configured to classify the lumen image as the first type or the second type using an image classification model generated by machine learning.

11. The endoscope control system according to claim 1, wherein the one or more processors are configured to determine a series of insertion operation details of the endoscope using the algorithm which is suitable for a situation around a distal end of the endoscope when the lumen image is classified as the second type.

12. The endoscope control system according to claim 11, wherein the one or more processors are configured to determine a completion of the determination of the series of insertion operation details of the endoscope using the algorithm based on the lumen image.

13. The endoscope control system according to claim 4, wherein the one or more processors are configured to determine
the insertion operation detail for changing a direction of a distal end of the endoscope to move the boundary of the bending portion near a center of the lumen image,
the insertion operation detail for advancing the distal end, and
the insertion operation detail for changing the direction of the distal end to a direction in which the lumen is estimated to exist after the distal end enters the bending portion.

14. The endoscope control system according to claim 5, wherein the one or more processors are configured to determine
the insertion operation detail for changing a direction of a distal end of the endoscope to move a lumen to a center of the lumen image,
the insertion operation detail for changing the direction of the distal end to move an obstacle that interferes with the advancement of the distal end to a direction toward an outside of the lumen image, and
the insertion operation detail for advancing the endoscope.

15. The endoscope control system according to claim 6, wherein the one or more processors are configured to determine
the insertion operation detail for inserting a distal end of the endoscope into one of a plurality of lumen candidate regions, and
the insertion operation detail for inserting the distal end into another lumen candidate region when the lumen candidate region into which the distal end is inserted is not the lumen.

16. A method of controlling an endoscope,
wherein one or more processors having hardware are configured to:
acquire an image of a lumen imaged by the endoscope;
classify the acquired lumen image as any one of a plurality of types;
determine the insertion operation detail of the endoscope using an insertion operation selection model generated by machine learning when the lumen image is classified as a first type; and
determine the insertion operation detail of the endoscope using an algorithm for determining the insertion operation detail when the lumen image is classified as a second type.

17. A storage medium comprising a program causing a computer to have a function of acquiring an image of a lumen imaged by an endoscope,
a function of classifying the acquired lumen image as any one of a plurality of types,
a function of determining an insertion operation detail of the endoscope using an insertion operation selection model generated by machine learning when the lumen image is classified as a first type, and
a function of determining the insertion operation detail of the endoscope using an algorithm for determining the insertion operation detail when the lumen image is classified as a second type.
